(19) United States
(12) Patent Application Publication
(10) Pub. No.: US 2018/0140572 A1
(43) Pub. Date: May 24, 2018

(54) COMPOSITIONS COMPRISING A CANCER STEMNESS INHIBITOR AND AN IMMUNOTHERAPEUTIC AGENT FOR USE IN TREATING CANCER

(71) Applicant: Boston Biomedical, Inc., Cambridge, MA (US)

(72) Inventors: Chiang J. Li, Cambridge, MA (US); Youzhi Li, Westwood, MA (US); Yuan Gao, Belmont, MA (US); Yuxin Wang, West Newton, MA (US); Janet Huang, Watertown, MA (US); Harry Rogoff, Wrentham, MA (US)

(21) Appl. No.: 15/579,013

(22) PCT Filed: Jun. 3, 2016
(86) PCT No.: PCT/US2016/035721

§ 371 Date: Dec. 1, 2017

Related U.S. Application Data
(60) Provisional application No. 62/170,498, filed on Jun. 3, 2015.
(60) Provisional application No. 62/233,081, filed on Sep. 25, 2015.

(51) Int. Cl. A61K 31/343; A61P 35/00; A61P 35/02; A61P 35/04; C07K 16/28; A61K 39/395; C07K 16/30

(52) U.S. Cl. CPC A61K 31/343; A61P 35/00; A61P 35/02; C07K 16/30; C07K 16/2818; A61K 39/3955; A61P 35/04

(57) ABSTRACT

Disclosed herein are methods for use in treating cancer comprising administering at least one cancer sternness inhibitor, for example, at least one STAT3 pathway inhibitor such as 2-acetylnaphtho [2, 3-b] furan-4, 9-dione, in order to sensitize or re-sensitive a cancer that is naive, resistant, or/and refractory to at least one immunotherapeutic agent, such as at least one immune checkpoint modulator.

The present application claims the benefit of priority under 35 U.S.C. § 119 of U.S. Provisional Patent Application Nos. 62/170,498, filed on Jun. 3, 2015, and 62/233,081, filed on Sep. 25, 2015.

Disclosed herein are methods for treating cancer in a subject comprising administering a therapeutically effective amount of at least one first compound chosen from cancer stemness inhibitors, prodrugs thereof, derivatives thereof, pharmaceutically acceptable salts of any of the foregoing, and solvates of any of the foregoing; and a therapeutically effective amount of at least one second compound chosen from immunotherapeutic agents, prodrugs thereof, derivatives thereof, pharmaceutically acceptable salts of any of the foregoing, and solvates of any of the foregoing.

In certain embodiments, the at least one first compound chosen from cancer stemness inhibitors is at least one compound of formula A chosen from compounds having formula A:

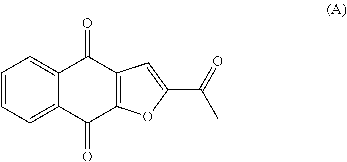 [SMILES: CC(=O)c1cc2c(o1)C(=O)c1ccccc1C2=O]

prodrugs thereof, derivatives thereof, pharmaceutically acceptable salts of any of the foregoing, and solvates of any of the foregoing.

In certain embodiments, the at least one second compound chosen from immunotherapeutic agents is at least one immune checkpoint modulator. In certain embodiments, the at least one second compound chosen from immunotherapeutic agents is at least one immune checkpoint modulator (e.g., an immune checkpoint inhibitor). In certain embodiments, the at least one immune checkpoint modulator (e.g., an immune checkpoint inhibitor) is chosen from nivolumab, pembrolizumab, ipilimumab, atezolizumab, durvalumab, lambrolizumab (MK3475), and tremelimumab. In certain embodiments, the at least one immune checkpoint modulator (e.g., an immune checkpoint inhibitor) is chosen from nivolumab, pembrolizumab, and ipilimumab.

The National Cancer Institute estimates 1,685,210 new cases of cancer will be diagnosed in the United States and 595,690 people will die from the disease in 2016. The most common cancers are projected to be breast cancer, lung and bronchus cancer, prostate cancer, colon and rectum cancer, bladder cancer, melanoma of the skin, non-Hodgkin lymphoma, thyroid cancer, kidney and renal pelvis cancer, leukemia, endometrial cancer, and pancreatic cancer. Despite advances in the treatment of certain forms of cancer through surgery, radiotherapy, and chemotherapy, many types of cancer are essentially incurable. Even when an effective treatment is available for a particular cancer, the side effects from the treatment can have a significant adverse impact on a patient's quality of life.

Most conventional chemotherapy agents have toxicity and limited efficacy, particularly for patients with advanced solid tumors. Conventional chemotherapeutic agents cause cytotoxicity to both healthy non-cancerous as well as cancerous cells. The therapeutic index of these chemotherapeutic compounds (i.e., a measure of the therapy's ability to distinguish between cancerous and normal cells) can be quite low. Frequently, a dose of a chemotherapy drug that is effective at killing cancer cells will also kill normal cells, especially those normal cells (such as epithelial cells and cells of the bone marrow) that undergo frequent cell division. When normal cells are subject to chemotherapy, side effects such as hair loss, suppression of hematopoiesis causing anemia and immunodeficiency, and nausea often occur. Depending on the general health of the patient, such side effects can preclude the administration of chemotherapy all together, or, at least, inflict significant discomfort on cancer patients that diminishes their quality of life. Even for cancer patients who respond to chemotherapy with tumor regression, cancers often quickly relapse, progress, and spread by metastasis after the initial response to chemotherapy. Such recurrent cancers are often highly refractory to additional rounds of chemotherapy treatment. As discussed below, cancer stem cells (CSCs) or cancer cells with high stemness (stemness-high cancer cells) are believed to be responsible for the rapid tumor recurrence and resistance observed after traditional chemotherapy.

CSCs are believed to possess at least the following four characteristics:
1. Stemness—As used herein, stemness means the capacity for a stem cell population to self-renew and transform into cancer cells (Gupta P B et al., Nat. Med. 2009; 15(9):1010-1012). While CSCs form only a small percentage of the total cancer cell population in a tumor (Clarke M F, Biol. Blood Marrow Transplant. 2009; 11(2 suppl. 2):14-16), they give rise to heterogeneous lineages of differentiated cancer cells that make up the bulk of the tumor (see Gupta et al. 2009). In addition, CSCs possess the ability to spread to other sites in the body by metastasis where they seed the growth of the new tumors (Jordan C T et al. N. Engl. J. Med. 2006; 355(12):1253-1261).
2. Aberrant signaling pathways—CSC stemness is associated with dysregulation of signaling pathways, which may contribute to their ability to metastasize. In normal stem cells, stemness signaling pathways are tightly controlled and genetically intact. In contrast, the aberrant regulation of sternness signaling pathways in CSCs plays a key role in the uncontrolled self-renewal of these cells and their transformation into cancer cells (see Ajani et al. 2015). Dysregulation of sternness signaling pathways also contributes to CSC resistance to chemotherapy and radiotherapy and to cancer recurrence and metastasis. Exemplary sternness signaling pathways involved in the induction and maintenance of sternness properties in CSCs include, but are not limited to, Janus kinase/signal transducers and activators of transcription (JAK/STAT), Hedgehog (Desert (DHH), Indian (IHH), and Sonic (SHH))/PATCHED/(PTCH1)/SMOOTHENED (SMO), NOTCH/DELTA-LIKE (DLL1, DLL3, DLL4)/JAGGED (JAG1, JAG2)/CSL (CBF1/Su(H)/Lag-1), WNT/APC/GSK3/β-CATENIN/TCF4 and NANOG (Boman B M et al., J. Clin. Oncol. 2008; 26(17):2828-2838).
3. Resistance to traditional therapies—Unfortunately, cancers that initially respond to chemotherapy and radiation treatment all too often relapse in a form that is resistant to these traditional therapies. While the detailed mechanism underlying such resistance is not well understood, aberrant regulation of CSC sternness signaling pathways (see Boman et al. 2008) in the context of a tumor's microenvironment (Borovski T. et al., Cancer Res. 2011; 71(3):634-639) may play a key role in the acquisition of such resistance.
4. Ability to contribute to tumor recurrence and metastasis—chemotherapy and radiation kills the majority of rapidly dividing cancer cells in a tumor but not CSCs that survive by acquiring resistance (see Jordan et al. 2006). Radiation/chemotherapy-resistant CSCs may also acquire the ability to metastasize to different sites in the body and maintain stemness at these locations through interactions with the microenvironment, thereby allowing for the spread of metastatic tumor growth (see Boman et al. 2008). Interestingly, this enhanced tumorigenicity of CSCs correlates with the expression of genes normally expressed in adult stem cells, such as cell surface markers like CD44, CD133, and CD166.

Because the survival of CSCs may be the principal reason why cancers relapse after treatment with chemotherapy and/or radiation, anti-cancer therapies that specifically target CSC's aberrant signaling pathways may help prevent tumor metastasis and provide a viable treatment option for patients with recurrent disease that is no longer treatable using traditional therapies. Such an approach may therefore improve the survival and quality of life of cancer patients, especially those patients suffering from metastatic disease. Unlocking this untapped potential involves the identification and validation of pathways that are essential for CSC self-renewal and survival. While many of the signaling pathways regulating embryonic or adult stem cell proliferation and differentiation are known, it remains to be seen if these same pathways are required for cancer stem cell self-renewal and survival.

The transcription factor Signal Transducer and Activator of Transcription 3 (also known as Acute-Phase Response Factor, APRF, DNA-Binding Protein APRF, ADMIO 3, HIES; referred to herein as STAT3) is a member of a family of seven transcription factors, STAT1 to STATE, including STAT5a and STAT5b. STATs are activated either by receptor associated tyrosine kinases like Janus kinases (JAKs) or by receptors with intrinsic tyrosine kinase activity such as PDGFR, EGFR, FLT3, EGFR, ABL, KDR, c-MET, or HER2. Upon tyrosine phosphorylation by receptor associated kinases, the phosphorylated STAT protein (“pSTAT”) dimerizes, as a homo- or heterodimer, and translocates from the cytoplasm to the nucleus, where it binds to specific DNA-response elements in the promoters of target genes and induces gene expression. STAT 2, 4, & 6 regulate primarily immune responses, while STAT3, along with STAT1 and STAT5, regulate the expression of genes controlling cell cycle (CYCLIN D1, D2, and c-MYC), cell survival (BCL-XL, BCL-2, MCL-1), and angiogenesis (HIF1α, VEGF) (Furqan et al. Journal of Hematology & Oncology (2013) 6:90).

In normal cells, STAT3 activation is transient and tightly regulated, lasting for example, from about 30 minutes to several hours. However, in a wide variety of human cancers, including all of the major carcinomas as well as some hematologic tumors, STAT3 is found to be aberrantly active. Persistently active STAT3 is present in more than half of all breast and lung cancers as well as colorectal cancer (CRC), ovarian cancer, hepatocellular carcinoma, multiple myeloma, and in more than 95% of all head/neck cancers. STAT3 therefore seems to play a pivotal role in cancer progression and may be one of the principal mechanisms by which cancer cells acquire drug resistance. STAT3 is a potent transcription regulator that targets genes involved in cell cycle, cell survival, oncogenesis, tumor invasion, and metastasis, including, but not limited to, BCL-XL, c-MYC, CYCLIN D1, VEGF, MMP-2, and SURVIVIN. STAT3 is also a key negative regulator of tumor immune surveillance and immune cell recruitment. Thus, STAT3 may enable the survival and self-renewal capacity of CSCs across a broad spectrum of cancers. A pharmaceutical compound with activity against CSCs, for example, through STAT3 inhibition, holds great promise as a treatment option for cancer patients with advanced disease.

In certain embodiments, the at least one compound of formula A is chosen from CSC growth and survival inhibitors. U.S. Pat. No. 8,877,803 describes a compound of formula A that inhibits STAT3 pathway activity with a cellular IC₅₀ of ˜0.25 μM. Example 13 in the '803 patent provides exemplary methods of synthesizing at least one compound of formula A. In certain embodiments, the at least one compound of formula A is used in a method for treating cancers. In Example 6 of PCT Patent Application No. PCT/US2014/033566 the at least one compound of formula A was chosen to enter a clinical trial for patients with advanced cancers. The disclosures of U.S. Pat. No. 8,877,803 and PCT Patent Application No. PCT/US2014/033566 are incorporated herein by reference in their entireties.

Immuno-oncology is a promising new area for cancer therapeutics. The immune system is capable of exquisite adaptation and selective targeting, a process that is now being harnessed and directed towards advanced cancer. Therapies in this field manipulate the immune response against cancer in a number of different ways. Vaccines have been developed with the goal of priming the cellular and humoral immune response towards specific cancer antigens, much in the same way as vaccines for microbiological diseases would do. Other therapies target the specific immune-evasion mechanisms that cancer cells use to avoid detection by the host immune system. These evasion mechanisms are the “checkpoints” of the immune system; specific cell-surface molecules that convince the immune effectors to spare the cells that express them. Recent clinical success with antibodies targeting programmed cell death-1 receptor (PD-1) and its ligands (PD-L1, PD-L2) has validated the concept that cancer cells can hijack immune checkpoint genes to subvert endogenous anti-cancer surveillance by the immune system. Ipilimumab, first approved in the United States in 2011, targets cytotoxic T-lymphocyte-associated antigen 4 (CTLA-4); while nivolumab and pembrolizumab, both of which were first approved in the United States in 2014, target PD-1 (Romano et al. (2015) J. Immunother. Cancer 2015; 3: 15).

In colorectal cancer, there is a strong association between the presence of tumor infiltrating lymphocytes (TILs) and disease prognosis. For example, a higher density of CD45RO⁺ memory T cells has been associated with longer overall survival and disease-free survival in patients with metastatic colorectal cancer. In another example, increased density of TILs in colorectal cancer liver metastases is associated with improved progression-free survival (PFS) rates.

Treatment strategies to augment TILs show promise; however, current FDA-approved checkpoint inhibitors have largely been unsuccessful in most gastrointestinal cancers. The anti-PD-1 and anti-PD-L1 antibodies showed no objective responses in unselected colorectal cancer patients. However, a recent Phase II study testing the PD-1 blockade on colorectal cancer reported that immune checkpoint inhibition could be beneficial in a cohort of patients with mismatch-repair deficiency indicating the higher somatic mutational load of the tumor cells may lead to higher neoantigen expression and recognition of the tumor cells by the immune system (Le et al., N. Engl. J. Med. (2015) 372: 2509-20). The objective response rate and 20-week progression-free survival rate in patients with mismatch repair deficient (dMMR) colorectal cancer were 40% and 78%, respectively, compared to 0% and 11% in mismatch repair proficient (pMMR) colorectal cancer (HR for disease progression or death, 0.10 [p<0.001], and HR for death, 0.22 [p=0.05]). Analysis of the tumor immune infiltrate at the invasive front showed a significantly greater density of CD8⁺ cytotoxic T cells in the dMMR versus the pMMR group (p=0.04). An increased intra-tumoral CD8⁺ T cell density was also significantly associated with an objective response (p=0.017). Whole exome sequencing and analysis of potential mutation associated neoantigens identified 1782 somatic mutations per tumor/578 neoantigens in the dMMR group compared to 73 mutations/21 neoantigen in the pMMR group. Therefore, although the effect of TILs is clearly associated with improved disease prognosis, current studies demonstrate that the benefit of overcoming the immune checkpoint may be limited to a small subset of colorectal cancer cells (e.g. ˜10-15%) with a high mutational burden. Moreover, pembrolizumab was reported to be not effective for patients with microsatellite stable (MSS) metastatic colorectal cancer (mCRC). For patients with MSS mCRC, the response rate was 0% and the disease control rate was 11%. The immune-related objective response rate and the immune-related progression-free survival at 20 weeks were 0% and 11%, respectively.

The present disclosure provides the surprising discovery that a treatment combination of at least one cancer stemness inhibitor and at least one immunotherapeutic agent, e.g. an immune checkpoint modulator, have a greater effect in inhibiting cancer cells than the added effects of both the at least one cancer stemness inhibitor and the at least one immunotherapeutic agent alone.

### BRIEF DESCRIPTION OF THE DRAWINGS

FIG. 1 shows an exemplary anti-tumor activity in a syngeneic mouse model of CT26 colon tumor of a control, an exemplary cancer stemness inhibitor, e.g., BBI608, an exemplary immunotherapeutic agent, e.g. an anti-PD-1 antibody, or an exemplary combination of a cancer stemness inhibitor and an immunotherapeutic agent, e.g. BBI-608 and an anti-PD1 antibody, according to certain embodiments of the present disclosure.

FIG. 2A and FIG. 2B show that an exemplary combination of a cancer stemness inhibitor and a checkpoint inhibitor, e.g., BBI608 and an anti-PD-1 antibody, resulted in a long-term anti-tumor memory in cured animals (CT26 models, FIG. 21A; 4T1 models, FIG. 21B) according to certain embodiments of the present disclosure.

FIG. 3A shows a bar graph that compared the number of spheres formed by CT26 xenograft colon cancer cells treated with either a control, an exemplary cancer stemness inhibitor, e.g., BBI608, an exemplary immunotherapeutic agent, e.g. an anti-PD-1 antibody, or an exemplary combination of a cancer stemness inhibitor and an immunotherapeutic agent, e.g. BBI-608 and an anti-PD1 antibody, according to certain embodiments of the present disclosure. FIG. 3B show an exemplary sphere formation study of CT26 xenograft colon cancer cells treated with a control, an exemplary cancer stemness inhibitor, e.g., BBI608, an exemplary immunotherapeutic agent, e.g. an anti-PD-1 antibody, or an exemplary combination of a cancer stemness inhibitor and an immunotherapeutic agent, e.g. BBI-608 and an anti-PD1 antibody, according to certain embodiments of the present disclosure.

FIG. 4 shows an exemplary expression of a cancer stemness marker protein, NANOG, in CT26 xenograft colon cancer cells treated with a control, an exemplary cancer stemness inhibitor, e.g., BBI608, an exemplary immunotherapeutic agent, e.g., an anti-PD-1 antibody, or an exemplary combination of a cancer stemness inhibitor and an immunotherapeutic agent, e.g., BBI608 and an anti-PD-1 antibody, according to certain embodiments of the present disclosure.

FIG. 5 shows the expression of exemplary cancer stemness marker proteins, e.g., CD133 and CD44, in CT26 xenograft colon cancer cells treated with a control, an exemplary cancer stemness inhibitor, e.g., BBI608, an exemplary immunotherapeutic agent, e.g., an anti-PD-1 antibody, or an exemplary combination of a cancer stemness inhibitor and an immunotherapeutic agent, e.g., BBI608 and an anti-PD-1 antibody, according to certain embodiments of the present disclosure.

FIG. 6 shows an exemplary reduction in expression of exemplary cancer stemness markers, e.g., β-CATENIN, NANOG, SMO, and SOX2, in cancer cells treated with an exemplary cancer stemness inhibitor, e.g., BBI608 (indicated by “*” in FIG. 6), according to certain embodiments of the present disclosure.

FIG. 7 shows an exemplary down-regulation of IL-6 protein production in HeLa cells treated with different concentrations of an exemplary cancer stemness inhibitor, e.g., BBI608 (indicated by “*” in FIG. 7), according to certain embodiments of the present disclosure.

FIG. 8 shows an exemplary down-regulation of IL-6, CYCLIN D1, MMP-9, and BLC2 gene expression in HeLa cells treated with an exemplary cancer stemness inhibitor, e.g., BBI608 (indicated by “*” in FIG. 8), according to certain embodiments of the present disclosure.

FIG. 9A shows an exemplary down-regulation of IL-6 protein production at 0, 1, 2, 4, 8, or 24 hours after treatment of SW480 xenograft colorectal cancer cells with an exemplary cancer stemness inhibitor, e.g., BBI608, according to certain embodiments of the present disclosure.

FIG. 9B shows an exemplary inhibition of CD44 protein expression at 0, 1, 2, 4, 8, 16, or 24 hours after treatment of SKOV3 xenograft ovarian cancer cells with an exemplary cancer stemness inhibitor, e.g., BBI608, according to certain embodiments of the present disclosure.

FIG. 10A and FIG. 10B show an exemplary reduction in IDO1 protein levels in SKOV3 xenograft ovarian cancer cells treated with an exemplary cancer sternness inhibitor, e.g., BBI608 (indicated by “*” in FIG. 10A and FIG. 10B), according to certain embodiments of the present disclosure.

FIG. 11 shows an exemplary reduction in interferon-gamma (IFNγ) induced IDO1 expression in SKOV3 xenograft ovarian cancer cells treated with an exemplary cancer sternness inhibitor, e.g., BBI608 (indicated by “*” in FIG. 11), according to certain embodiments of the present disclosure.

FIG. 12A and FIG. 12B show an exemplary reduction in interferon-gamma (IFNγ) induced IDO1 expression in HeLa cells treated with an exemplary cancer sternness inhibitor, e.g., BBI608 (indicated by “*” in FIG. 12A and FIG. 12B), according to certain embodiments of the present disclosure.

FIG. 13A and FIG. 13B show an exemplary reduction in IDO1 expression at 0, 1, 2, 4, 8, and 24 hours after treatment of SW480 xenograft colorectal cancer cells (FIG. 13A) and SKOV3 xenograft ovarian cancer cells (FIG. 13B) with an exemplary cancer sternness inhibitor, e.g., BBI608, according to certain embodiments of the present disclosure.

FIG. 14 shows an exemplary expression of the checkpoint molecule PD-L1 in cancer cells treated with a control, an exemplary checkpoint inhibitor, e.g., an anti-PD-1 antibody, an exemplary cancer sternness inhibitor, e.g., BBI608, or an exemplary combination of a checkpoint inhibitor with a cancer sternness inhibitor, e.g. an anti-PD-1 antibody and BBI-608, according to certain embodiments of the present disclosure.

FIG. 15A shows an exemplary down-regulation of IFNγ-induced PD-L1 expression in cancer cells treated with an exemplary cancer sternness inhibitor, e.g., BBI608, according to certain embodiments of the present disclosure.

FIG. 15B shows an exemplary down-regulation of PD-L1 expression in vivo after treatment with an exemplary cancer stemness inhibitor, e.g., BBI608, according to certain embodiments of the present disclosure.

FIG. 16 shows that an exemplary cancer stemness inhibitor, e.g. BBI608, increased B-cell activation in vivo according to certain embodiments of the present disclosure.

FIG. 17 shows that an exemplary cancer stemness inhibitor, e.g., BBI608, increased proliferating CD8⁺ T cells in an Apc^{Min/+} mouse model of colon cancer according to certain embodiments of the present disclosure.

FIG. 18 shows that treatment of cancer with an exemplary combination of cancer stemness inhibitor and a checkpoint inhibitor, e.g., BBI608 and an anti-PD-1 antibody, increased the number of tumor infiltrating T lymphocytes present in a tumor sample, as indicated by CD3 antibody immunofluorescence staining, according to certain embodiments of the present disclosure.

FIG. 19A shows that treatment of cancer with an exemplary combination of cancer stemness inhibitor and a checkpoint inhibitor, e.g., BBI608 and an anti-PD-1 antibody, increased the number of tumor infiltrating T lymphocytes (TILs) present in a tumor sample as compared to treatment with either BBI608 or the anti-PD-1 antibody alone according to certain embodiments of the present disclosure.

FIG. 19B shows that treatment of a mouse cancer model with an exemplary combination of cancer stemness inhibitor and a checkpoint inhibitor, e.g., BBI608 and an anti-PD-1 antibody, increased the percentage of CD3⁺ tumor infiltrating T lymphocytes (TILs) amongst the total number of cells present in a tumor sample as compared to treatment with either BBI608 or the anti-PD-1 antibody alone according to certain embodiments of the present disclosure.

FIG. 19C shows that treatment of a mouse cancer model with an exemplary combination of cancer stemness inhibitor and a checkpoint inhibitor, e.g., BBI608 and the anti-PD-1 antibody, increased the percentage of CD8⁺ tumor infiltrating T lymphocytes (TILs) amongst the total number of cells present in a tumor sample as compared to treatment with either BBI608 or the anti-PD-1 antibody alone according to certain embodiments of the present disclosure.

FIG. 20 shows that an exemplary cancer stemness inhibitor, e.g., BBI608, increased the number of IFN-γ producing tumor-specific cytotoxic T cells in a tumor isolated from a ApcMin/+ mouse model of colon cancer according to certain embodiments of the present disclosure.

The following are definitions of terms used in the present specification.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

When a range of values is listed herein, it is intended to encompass each value and sub-range within that range. For example, “1-5 mg” is intended to encompass 1 mg, 2 mg, 3 mg, 4 mg, 5 mg, 1-2 mg, 1-3 mg, 1-4 mg, 1-5 mg, 2-3 mg, 2-4 mg, 2-5 mg, 3- 4 mg, 3-5 mg, and 4-5 mg.

The terms “administer,” “administering,” or “administration” are used herein in their broadest sense. These terms refer to any method of introducing to a subject a compound or pharmaceutical composition described herein and can include, for example, introducing a compound systemically, locally, or in situ to the subject. Thus, a compound of the present disclosure produced in a subject from a composition (whether or not it includes the compound) is encompassed by these terms. When these terms are used in connection with the term “systemic” or “systemically,” they generally refer to in vivo systemic absorption or accumulation of the compound or composition in the blood stream and its distribution throughout the entire body.

The term “cancer” in a subject refers to the presence of cells possessing characteristics typical of cancer-causing cells, such as uncontrolled proliferation, immortality, metastatic potential, rapid growth and proliferation rate, and certain morphological features. Often, cancer cells will be in the form of a tumor or mass, but such cells may exist alone within a subject, or may circulate in the blood stream as independent cells, such as leukemic or lymphoma cells. Examples of cancer as used herein include, but are not limited to, lung cancer, pancreatic cancer, bone cancer, skin cancer, head or neck cancer, cutaneous or intraocular melanoma, breast cancer, uterine cancer, ovarian cancer, peritoneal cancer, colon cancer, rectal cancer, colorectal adenocarcinoma, cancer of the anal region, stomach cancer, gastric cancer, gastrointestinal cancer, gastric adenocarcinoma, adrenocorticoid carcinoma, uterine cancer, carcinoma of the fallopian tubes, carcinoma of the endometrium, carcinoma of the vagina, carcinoma of the vulva, Hodgkin's Disease, esophageal cancer, gastroesophageal junction cancer, gastroesophageal adenocarcinoma, chondrosarcoma, cancer of the small intestine, cancer of the endocrine system, cancer of the thyroid gland, cancer of the parathyroid gland, cancer of the adrenal gland, sarcoma of soft tissue, Ewing's sarcoma, cancer of the urethra, cancer of the penis, prostate cancer, bladder cancer, testicular cancer, cancer of the ureter, carcinoma of the renal pelvis, mesothelioma, hepatocellular cancer, biliary cancer, kidney cancer, renal cell carcinoma, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. Some of the exemplified cancers are included in general terms and are included in this term. For example, urological cancer, a general term, includes bladder cancer, prostate cancer, kidney cancer, testicular cancer, and the like; and hepatobiliary cancer, another general term, includes liver cancers (itself a general term that includes hepatocellular carcinoma or cholangiocarcinoma), gallbladder cancer, biliary cancer, or pancreatic cancer. Both urological cancer and hepatobiliary cancer are contemplated by the present disclosure and included in the term “cancer.”

Also included within the term “cancer” is the term “solid tumor” or “advanced solid tumor.” A “solid tumor” refers to those conditions, such as cancer, that form an abnormal tumor mass, such as sarcomas, carcinomas, and lymphomas. Examples of solid tumors include, but are not limited to, non-small cell lung cancer (NSCLC), neuroendocrine tumors, thyomas, fibrous tumors, metastatic colorectal cancer (mCRC), and the like. In certain embodiments, the solid tumor disease is an adenocarcinoma, squamous cell carcinoma, large cell carcinoma, and the like.

In certain embodiments, the cancer is esophageal cancer, gastroesophageal junction cancer, gastroesophageal adenocarcinoma, gastric cancer, chondrosarcoma, colorectal adenocarcinoma, breast cancer, ovarian cancer, head and neck cancer, melanoma, gastric adenocarcinoma, lung cancer, pancreatic cancer, renal cell carcinoma, hepatocellular carcinoma, cervical cancer, brain tumor, multiple myeloma, leukemia, lymphoma, prostate cancer, cholangiocarcinoma, endometrial cancer, small bowel adenocarcinoma, uterine sarcoma, or adrenocorticoid carcinoma. In certain embodiments, the cancer is esophageal cancer, gastroesophageal junction cancer, gastroesophageal adenocarcinoma, colorectal adenocarcinoma, breast cancer, ovarian cancer, head and neck cancer, melanoma, gastric adenocarcinoma, lung cancer, pancreatic cancer, renal cell carcinoma, hepatocellular carcinoma, cervical cancer, brain tumor, multiple myeloma, leukemia, lymphoma, prostate cancer, cholangiocarcinoma, endometrial cancer, small bowel adenocarcinoma, uterine sarcoma, or adrenocorticoid carcinoma. In certain embodiments, the cancer is breast cancer. In certain embodiments, the cancer is colorectal adenocarcinoma. In certain embodiments, the cancer is small bowel adenocarcinoma. In certain embodiments, the cancer is hepatocellular carcinoma. In certain embodiments, the cancer is head and neck cancer. In certain embodiments, the cancer is renal cell carcinoma. In certain embodiments, the cancer is ovarian cancer. In certain embodiments, the cancer is prostate cancer. In certain embodiments, the cancer is lung cancer. In certain embodiments, the cancer is uterine sarcoma. In certain embodiments, the cancer is esophageal cancer. In certain embodiments, the cancer is endometrial cancer. In certain embodiments, the cancer is cholangiocarcinoma. In certain embodiments, each of the cancers is unresectable, advanced, refractory, recurrent, or metastatic.

In certain embodiments, the cancer is esophageal cancer, gastroesophageal junction cancer, lung cancer, gastrointestinal cancer, leukemia, lymphoma, myeloma, brain cancer, pancreatic cancer, prostate cancer, liver cancer, gastroesophageal adenocarcinoma, chondrosarcoma, colorectal adenocarcinoma, microsatellite instability-high metastatic colorectal cancer, microsatellite stable metastatic colorectal cancer, colorectal cancer with mismatch-repair deficiency, colorectal cancer without mismatch-repair deficiency, breast cancer, ovarian cancer, head and neck cancer, melanoma, gastric adenocarcinoma, sarcoma, genitourinary cancer, gynecologic cancer, or adrenocorticoid carcinoma.

In certain embodiments, the efficacy of a compound or a combination of compounds is tested in a xenograft cancer model in which cells isolated from a solid tumor are injected into a host animal, e.g. an immunocompromised host, to establish solid tumors. In certain embodiments, the cells isolated from a solid tumor comprise cancer stem cells. The host animal can be a model organism such as nematode, fruit fly, zebrafish, or a laboratory mammal such as a mouse (nude mouse, SCID mouse, NOD/SCID mouse, Beige/SCID Mouse), rat, rabbit, or primate. The severely immunodeficient NOD-SCID mice may be chosen as recipients to maximize the participation of injected cells. Immunodeficient mice do not reject human tissues, and SCID and NOD-SCID mice have been used as hosts for in vivo studies of human hematopoiesis and tissue engraftment. McCune et al., Science 241: 1632-9 (1988); Kamel-Reid & Dick, Science 242: 1706-9 (1988); Larochelle et al., Nat. Med. 2: 1329-37 (1996). In addition, Beige/SCID mice also have been used.

In certain embodiments, the efficacy of a compound or a combination of compounds is tested in a syngeneic cancer model in which cells isolated from a solid tumor are injected into a host animal, e.g. an immunocompetent host, to establish solid tumors. In certain embodiments, the cells isolated from a solid tumor comprise cancer stem cells. The host animal can be a model organism such as nematode, fruit fly, zebrafish; preferably a laboratory mammal such as a mouse (C57BL/6, BALB/c, VM/Dk, and B6D2F1 Mouse), rat, rabbit, or primate.

As used herein, the term “cancer stemness inhibitor” means a molecule that can target, reduce, inhibit, interfere with, or modulate at least one of a plurality of pathways involved in cancer stemness or the expression (e.g., the production of a functional product, e.g., a protein) of at least one of a plurality of cancer stemness genes. The expression or the expressed proteins can be used as biomarkers of the corresponding cancer stemness genes. Examples of these biomarkers include, but are not limited to, β-CATENIN, NANOG, SMO, SOX2, STAT3, AXL, ATM, c-MYC, KLF4, SURVIVIN, or BMI-1. A cancer stemness inhibitor may alter cancer stem cell growth as well as heterogeneous cancer cell growth.

In certain embodiments, a cancer stemness inhibitor is a small molecule that binds a protein encoded by a cancer stemness gene. In certain embodiments, a cancer stemness inhibitor is a biologic, e.g., a recombinant binding protein or peptide (e.g. APTSTAT3; see Kim et al. Cancer Res. (2014) 74(8):2144-51) or nucleic acid (e.g. STAT3 aiRNA; see U.S. Pat. No. 9,328,345, the content of which is incorporated herein in its entirety), or conjugate thereof, that binds to a protein encoded by a cancer stemness gene. In certain embodiments, a cancer stemness inhibitor is a cell. In certain embodiments, a cancer stemness inhibitor is a STAT3 inhibitor (for example, that binds to and inhibits a biological activity of STAT3 (see Furtek et al., ACS Chem. Biol., 2016, 11 (2), pp 308-318)).

In certain embodiments, a cancer stemness inhibitor is at least one compound chosen from 2-(1-hydroxyethyl)-naphtho[2,3-b]furan-4,9-dione, 2-acetyl-7-chloro-naphtho[2,3-b]furan-4,9-dione, 2-acetyl-7-fluoro-naphtho[2,3-b]furan-4,9-dione, 2-acetylnaphtho[2,3-b]furan-4,9-dione, or 2-ethyl-naphtho[2,3-b]furan-4,9-dione, prodrugs thereof, derivatives thereof, pharmaceutically acceptable salts of any of the foregoing, and solvates of any of the foregoing.

In certain embodiments, a cancer stemness inhibitor of the present disclosure may be administered in an amount ranging from about 300 mg to about 700 mg. In certain embodiments, the cancer stemness inhibitor may be administered in an amount ranging from about 700 mg to about 1200 mg. In certain embodiments, the cancer stemness inhibitor may be administered in an amount ranging from about 800 mg to about 1100 mg. In certain embodiments, the cancer stemness inhibitor may be administered in an amount ranging from about 850 mg to about 1050 mg. In certain embodiments, the cancer stemness inhibitor may be administered in an amount ranging from about 960 mg to about 1000 mg.

In certain embodiments, the total amount of the cancer stemness inhibitor is administered once daily. In certain embodiments, the cancer stemness inhibitor is administered in a dose of about 480 mg daily. In certain embodiments, the cancer stemness inhibitor is administered in a dose of about 960 mg daily. In certain embodiments, the cancer stemness inhibitor is administered in a dose of about 1000 mg daily. In certain embodiments, the total amount of the cancer stemness inhibitor is administered in divided doses more than once daily, such as twice daily (BID) or more often. In certain embodiments, the cancer stemness inhibitor is administered in a dose of about 240 mg twice daily. In certain embodiments, the cancer stemness inhibitor is administered in a dose of about 480 mg twice daily. In certain embodiments, the cancer stemness inhibitor is administered in a dose of about 500 mg twice daily. In certain embodiments, the cancer stemness inhibitor is administered orally.

In certain embodiments, a cancer stemness inhibitor is at least one compound of formula A.

As used herein, the terms “at least one compound of formula A” and “Compound A” each means a compound chosen from compounds having formula A:

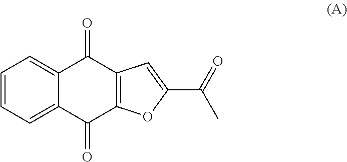 [SMILES: CC(=O)c1cc2c(o1)C(=O)c1ccccc1C2=O]

prodrugs thereof, derivatives thereof, pharmaceutically acceptable salts of any of the foregoing, and solvates of any of the foregoing.

In certain embodiments, prodrugs and derivatives of compounds having formula A are STAT3 inhibitors. Non-limiting examples of prodrugs of compounds having formula A include, for example, the phosphoric ester and phosphoric diester described in U.S. pre-grant Publication No. 2012/0252763 as compound numbers 4011 and 4012 and also suitable compounds described in in U.S. Pat. No. 9,150,530. Non-limiting examples of derivatives of compounds having formula A include, for example, the derivatives disclosed in U.S. Pat. No. 8,977,803. The disclosures of U.S. pre-grant Publication No. 2012/0252763 and U.S. Pat. Nos. 9,150,530 and 8,977,803 are incorporated herein by reference in their entireties.

Compounds having formula A, shown below,

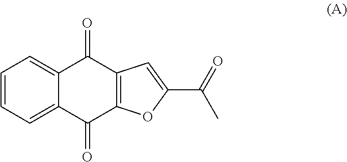 [SMILES: CC(=O)c1cc2c(o1)C(=O)c1ccccc1C2=O]

may also be known as 2-acetylnaphtho[2,3-b]furan-4,9-dione, napabucasin, or BBI608, and include tautomers thereof.

Suitable methods of preparing 2-acetylnaphtho[2,3-b]furan-4,9-dione, including its crystalline forms, and additional cancer stemness inhibitors are described in the co-owned PCT applications published as WO 2009/036099, WO 2009/036101, WO 2011/116398, WO 2011/116399, and WO 2014/169078. The contents of each of these applications are incorporated herein by reference in their entireties.

In certain embodiments, the at least one compound of formula A may be administered in an amount ranging from about 80 mg to about 1500 mg. In certain embodiments, the at least one compound of formula A may be administered in an amount ranging from about 160 mg to about 1000 mg. In certain embodiments, the at least one compound of formula A may be administered in an amount ranging from about 300 mg to about 700 mg a day. In certain embodiments, the at least one compound of formula A may be administered in an amount ranging from about 700 mg to about 1200 mg. In certain embodiments, the at least one compound of formula A may be administered in an amount ranging from about 800 mg to about 1100 mg. In certain embodiments, the at least one compound of formula A may be administered in an amount ranging from about 850 mg to about 1050 mg. In certain embodiments, the at least one compound of formula A may be administered in an amount ranging from about 960 mg to about 1000 mg. In certain embodiments, the total amount of the at least one compound of formula A is administered once daily. In certain embodiments, the at least one compound of formula A is administered in a dose of about 480 mg daily. In certain embodiments, the at least one compound of formula A is administered in a dose of about 960 mg daily. In certain embodiments, the at least one compound of formula A is administered in a dose of about 1000 mg daily. In certain embodiments, the total amount of the at least one compound of formula A is administered in divided doses more than once daily, such as twice daily (BID) or more often. In certain embodiments, the at least one compound of formula A may be administered in an amount ranging from about 80 mg twice daily to about 750 mg twice daily. In certain embodiments, the at least one compound of formula A may be administered in an amount ranging from about 80 mg twice daily to about 500 mg twice daily. In certain embodiments, the at least one compound of formula A is administered in a dose of about 240 mg twice daily. In certain embodiments, the at least one compound of formula A is administered in a dose of about 480 mg twice daily. In certain embodiments, the at least one compound of formula A is administered in a dose of about 500 mg twice daily. In certain embodiments, the at least one compound of formula A is administered orally.

The terms “combination,” “combinatorial,” or “treatment combination,” as used herein, mean the administration of at least two different agents (e.g., at least one first compound chosen from cancer stemness inhibitors and/or at least one second compound chosen from immunotherapeutic agents, and, optionally, one or more additional agents) to treat a disorder, condition, or symptom, e.g., a cancer condition. Such combination/treatment combination may involve the administration of one agent before, during, and/or after the administration of a second agent. The first agent and the second agent can be administered concurrently, separately, or sequentially to a subject in separate pharmaceutical compositions. The first agent and the second agent may be administered to a subject by the same or different routes of administration. In certain embodiments, a treatment combination comprises a therapeutically effective amount of at least one first compound chosen from cancer stemness inhibitors and a therapeutically effective amount of at least one second compound chosen from immunotherapeutic agents.

For example, the at least one first compound chosen from cancer stemness inhibitors and the at least one second compound chosen from immunotherapeutic agents can have different mechanisms of action. In certain embodiments, a treatment combination improves the prophylactic or therapeutic effect of the at least one first compound chosen from cancer stemness inhibitors and the at least one second compound chosen from immunotherapeutic agents by functioning together to have an additive, synergistic, or enhanced effect. In certain embodiments, a treatment combination of the present disclosure reduces the side effects associated with the at least one first compound chosen from cancer stemness inhibitors or the at least one second compound chosen from immunotherapeutic agents. The administration of the at least one first compound chosen from cancer stemness inhibitors and the at least one second compound chosen from immunotherapeutic agents may be separated in time by up to several weeks, but more commonly within 48 hours, and most commonly within 24 hours.

The terms “effective amount” and “therapeutically effective amount” refer to that amount of a compound or pharmaceutical composition described herein that is sufficient to produce an intended result including, but not limited to, disease treatment, as illustrated below. In certain embodiments, the “therapeutically effective amount” refers to the amount of a compound or pharmaceutical composition that is administered systemically, locally, or in situ (e.g., the amount of compound that is produced in situ in a subject). The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art, e.g., a board-certified oncologist. The term also applies to a dose that will induce a particular response in target cells, e.g., reduction of cell migration. The specific dose may vary depending on, for example, the weight of the subject, the particular pharmaceutical composition, the subject and their age and existing health conditions or risk for health conditions, the dosing regimen to be followed, the severity of the disease, whether it is administered in combination with other agents, the timing of administration, the tissue to which it is administered, and the physical delivery system in which it is carried.

An “effective amount” of an anti-cancer agent in reference to decreasing cancer cell growth, means an amount capable of decreasing, to some extent, the growth of some cancer or tumor cells. The term includes an amount capable of invoking a growth inhibitory, cytostatic and/or cytotoxic effect, and/or apoptosis of the cancer or tumor cells.

A “therapeutically effective amount” in reference to the treatment of a subject's cancer, means an amount capable of invoking, for example, one or more of the following effects: (1) inhibition, to some extent, of cancer or tumor growth, including a decrease or cessation in the progression of the subject's cancer; (2) reduction in the number of cancer or tumor cells;

(3) reduction in tumor size; (4) inhibition, e.g., a decrease or a cessation, of cancer or tumor cell infiltration into peripheral organs; (5) inhibition, e.g., a decrease or a cessation, of metastasis;
(6) enhancement of anti-tumor immune response, which may, but is not required to, result in the regression or rejection of the tumor, or (7) relief, to some extent, of one or more symptoms associated with the cancer or tumor. The therapeutically effective amount may vary according to factors such as the disease state, age, sex, and weight of the individual and the ability of one or more anti-cancer agents to elicit a desired response in the individual. A “therapeutically effective amount” is an amount of a compound where any toxic or detrimental effects resulting from the administration of the compound are outweighed by the therapeutically beneficial effects.

The term “immunotherapeutic agent,” as used herein, refers to any agent that can induce, enhance, or suppress an immune response in a subject. In certain embodiments, an immunotherapeutic agent can be an immune checkpoint modulator. As used herein, the term “immune checkpoint modulator” refers to a molecule that can completely or partially reduce, inhibit, interfere with, or modulate one or more immune checkpoint proteins that regulate T-cell activation or function. In certain embodiments, the immune checkpoint modulator is an immune checkpoint inhibitor.

Non-limiting examples of immune checkpoint proteins include cytotoxic T-lymphocyte-associated antigen (CTLA, for example, CTLA4) and its ligands CD 80 and CD86; programmed cell death protein (PD, for example, PD-1) and its ligands PDL1 and PDL2; indoleamine-pyrrole 2,3-dioxygenase-1 (IDO1); T cell membrane protein (TIM, for example, TIM3); adenosine A2a receptor (A2aR); lymphocyte activation gene (LAG, for example, LAG3); killer immunoglobulin receptor (KIR); and the like. These proteins are responsible for co-stimulatory or inhibitory T-cell responses. Immune checkpoint proteins regulate and maintain self-tolerance and the duration and amplitude of physiological immune responses.

In certain embodiments, an immune checkpoint modulator (e.g., an immune checkpoint inhibitor) can be a small molecule, an antibody, a recombinant binding protein, or a peptide that binds to or inhibits a biological activity of an immune checkpoint protein.

Non-limiting examples of immune checkpoint modulators (e.g., immune checkpoint inhibitors) include CTLA4 inhibitors, PD1 inhibitors, PDL1s, LAG3 inhibitors, KIR inhibitors, B7-H3 ligands, B7-H4 ligands, and TIM3 inhibitors.

In certain embodiments, an immunotherapeutic agent is chosen from, for example, AMP-224 (a recombinant B7-DC Fc-fusion protein composed of the extracellular domain of the PD-1 ligand programmed cell death ligand 2 (PD-L2, B7-DC) and the Fc region of human immunoglobulin (Ig) G1 that binds to PD-1 (the recombinant fusion protein is also referred to as B7-DC1g; see, for example, the International Patent Application Nos. PCT/US2009/054969 and PCT/US2010/057940, the contents of which are hereby incorporated herein in their entireties)), alemtuzumab (that binds to CD52 (alemtuzumab is also referred to as, Campath, MabCampath, Lemtrada, Campath-1H, LDP-03; see, for example, U.S. Pat. Nos. 5,846,534, 7,910,104, 8,440,190, 8,623,357, and 8,617,554, the contents of which are hereby incorporated herein in their entireties)), atezolizumab (that binds to PD-L1 (atezolizumab is also referred to as MPDL3280A, RG7446, YW243.55.S70; see, for example, U.S. Pat. No. 8,217,149, the content of which is hereby incorporated herein in its entirety)), bavituximab (that binds to phosphatidylserine (bavituximab is also referred to as ch3G4; see, for example, U.S. Pat. No. 7,572,442, the content of which is hereby incorporated herein in its entirety)), bevacizumab (that binds to VEGF-A (bevacizumab is also referred to as Avastin, Altuzan, rhuMab-VEGF, RG435, A4.6.1; see, for example, U.S. Pat. Nos. 7,169,901, 7,691,977, 7,758,859, and 8,101,177, the contents of which are hereby incorporated herein in their entireties)), BMS-936559 (that binds the programmed cell death-1 ligand 1 (PD-L1) (the BMS-936559 antibody is also referred to as MDX-1105 or 12A4; see, for example, U.S. Pat. Nos. 7,943,743, 9,102,725, and 9,212,224, the contents of which are hereby incorporated herein in their entireties)), BMS-986016 (that binds to LAG3 (CD223) (the BMS-986016 antibody is also referred to as 25F7 or BMS 986016; see, for example, the published U.S. Patent Application No. 2015/0307609, the content of which is hereby incorporated herein in its entirety)), brentuximab vedotin (a chimeric human/mouse antibody drug conjugate that binds to CD30 (brentuximab is also referred to as Adcetris, SGN-35, cAC10-vcMMAE, AC10; see, for example, U.S. Pat. No. 7,090,843, the content of which is hereby incorporated herein in its entirety)), cetuximab (that binds to EGFR (cetuximab is also referred to as Erbitux, IMC-C225, CMAB009, Mab C225; see, for example, the International PCT Application No. PCT/US2015/050131 and the published U.S. Patent Application No. 2015/0307609, the contents of which are hereby incorporated herein in their entireties)), gemtuzumab ozogamicin (a humanized mouse antibody drug conjugate that binds to CD33 (gemtuzumab ozogamicin is also referred to as Mylotarg, CMA-676, P67.6; see, for example, the published U.S. Patent Application No. 2007/0009532, the content of which is hereby incorporated herein in its entirety)), durvalumab (that binds to PD-L1 (durvalumab is also referred to as MEDI-4736, MEDI4736; see, for example, U.S. Pat. No. 8,779,108 and the published U.S. Patent Application 2016/006,0344, the contents of which are hereby incorporated herein in their entireties)), ibritumomab tiuxetan (a murine IgG1 monoclonal antibody conjugated to the chelator tiuxetan, that binds to CD20 (ibritumomab tiuxetan is also referred to as Zevalin, 2B8, C2B8, Y2B8; see, for example, U.S. Pat. No. 7,422,739, the content of which is hereby incorporated herein in its entirety)), IMP321 (a 200 kDA soluble dimeric recombinant fusion protein of the extracellular portion of LAG3 with immunoglobulin, (see, for example, the published U.S. Patent Application No 2011/008331, the content of which is hereby incorporated herein in its entirety)), ipilimumab (that binds to CTLA4 (ipilimumab is also referred to as Yervoy, MDX-010, MDX101, 10D1, BMS-734016; see, for example, U.S. Pat. Nos. 6,984,720, 8,784,815, and 8,685,394, the contents of which are hereby incorporated herein in their entireties)), lirilumab (that binds to Killer-cell immunoglobulin-like receptors (KIRs) (lirilumab is also referred to as IPH 2101, IPH2101, 1-7F9, IPH 2102, IPH2102 or BMS-986015; see, for example, U.S. Pat. Nos. 8,119,775 and 8,981,065, the contents of which are hereby incorporated herein in their entireties)), enoblituzumab (a humanized mouse antibody that binds to B7-H3 (enoblituzumab is also referred to as MGA271; see, for example, U.S. Pat. Nos. 8,802,091 and 9,150,656, the contents of which are hereby incorporated herein in their entireties)), nivolumab (that binds to PD-1 (nivolumab is also referred to as Opdivo, ONO-4538, MDX-1106, BMS-936558, 5C4; see, for example, U.S. Pat. Nos. 8,008,449, 9,084,776, and 8,168,179, the contents of which are hereby incorporated herein in their entireties)), ofatumumab (that binds to CD20 (ofatumumab is also referred to as Arzerra, GSK1841157, HuMax-CD20, 2F2; see, for example, U.S. Pat. No. 8,529,902, the content of which is hereby incorporated herein in its entirety)), panitumumab (that binds EGFR (panitumumab is also referred to as Vectibix, ABX-EGF, clone E7.6.3, Pmab, 139; see, for example, U.S. Pat. Nos. 6,235,883, 7,807,798, 9,062,113, and 9,096,672, the contents of which are hereby incorporated herein in their entireties)), pembrolizumab (that binds to PD-1 (pembrolizumab is also referred to as Keytruda, MK-3475, SCH 900475, lambrolizumab; see, for example, U.S. Pat. Nos. 8,354,509, 9,220,776, 8,952,136, and 8,900,587, the contents of which are hereby incorporated herein in their entireties)), pidilizumab (that binds to CD20 (pidilizumab is also referred to as Arzerra, GSK1841157, HuMax-CD20, 2F2 or CT-011; see, for example, U.S. Pat. No. 8,529,902, the content of which is hereby incorporated herein in its entirety)), rituximab (that binds to CD20 (rituximab is also referred to as MabThera, Rituxan, C2B8, IDEC-C2B8, IDEC-102 or RG105; see, for example, U.S. Pat. No. 5,736,137, the content of which is hereby incorporated herein in its entirety)), tositumomab (that binds to CD20 (tositumomab is also referred to as Bexxar, or 1131; see, for example, U.S. Pat. No. 5,595,721, the content of which is hereby incorporated herein in its entirety)), trastuzumab (that binds to HER2/neu (trastuzumab is also referred to as Herceptin, RG597, anti-p185-HER2, huMAb4D5-8, rhuMAb HER2; see, for example, U.S. Pat. Nos. 7,435,797 and 7,560,111, the contents of which are hereby incorporated herein in their entireties)), tremelimumab (that binds to CTLA4 (tremelimumab is also referred to as ticilimumab, CP-675206, clone 11.2.1; see, for example, U.S. Pat. Nos. 6,682,736, 8,685,394, 7,824,679, and 8,143,379, the contents of which are hereby incorporated herein in their entireties)), and urelumab (that binds to 4-1BB (urelumab is also referred to as BMS-663513; see, for example, U.S. Pat. No. 8,716,452, the content of which is hereby incorporated herein in its entirety)).

In certain embodiments, the immunotherapeutic agent is chosen from atezolizumab (MPDL3280A), durvalumab, ipilimumab, lambrolizumab (MK3475), nivolumab, pembrolizumab, or tremelimumab (MEDI4736). In certain embodiments, the immunotherapeutic agent is chosen from ipilimumab, nivolumab, and pembrolizumab.

In certain embodiments, ipilimumab can be administered, e.g., at a dose of about 3 mg/kg intravenously over about 90 minutes once every 3 weeks for a total of 4 doses. In certain embodiments, pembrolizumab is administered, e.g., at a dose of about 2 mg/kg intravenously over about 30 minutes once every 3 weeks. In certain embodiments, nivolumab is administered, e.g., at a dose of about 3 mg/kg intravenously over about 60 minutes once every 2 weeks.

In certain embodiments, the immunotherapeutic agent is a cytokine, for example, an interferon (IFN), interleukin, or the like. Specifically, the immunotherapeutic agent can be interferon (IFNα or IFNβ), type 2 (IFNγ), or type III (IFNλ). The immunotherapeutic agent can also be, for example, interleukin-1 (IL-1), interleukin-1α (IL-1α), interleukin-1β 4-1β), interleukin-2 (IL-2), interleukin-3 (IL-3), interleukin-4 (IL-4), interleukin-6 (IL-6), interleukin-8 (IL-8), interleukin-10 (IL-10), interleukin-11 (IL-11), interleukin-12 (IL-12), interleukin-13 (IL-13), or interleukin-18 (IL-18), or the like.

In certain embodiments, the immunotherapeutic agent can be a cell, for example, an immune cell. For example, an immune cell, e.g., one that is specific to a tumor, can be activated, cultured, and administered to a patient. The immune cell can be a natural killer cell, lymphokine-activated killer cell, cytotoxic T-cell, dendritic cell, or a tumor infiltrating lymphocyte (TIL). In certain embodiments, the immunotherapeutic agent can be sipuleucel-T (APC8015, Provenge™).

As used herein, “tumor infiltrating lymphocytes” (“TILs”) refer to white blood cells (i.e., T cells, B cells, NK cells, macrophages) that have left the bloodstream and migrated into a tumor. An analysis of patients with metastatic gastrointestinal cancers suggests CD4⁺ and CD8⁺ T cells within the TIL population are able to recognize neo-epitopes derived from somatic mutations expressed by the patient's tumor.

The terms “progress,” “progressed,” and “progression” as used herein refer to at least one of the following: (1) a response to prior therapy (e.g., chemotherapy) of progressive disease (PD); (2) the appearance of one or more new lesions after treatment with prior therapy (e.g., chemotherapy); and (3) at least a 5% (e.g., 10%, 20%) increase in the sum of diameters of target lesions, taking as a reference the smallest sum on study (this includes the baseline sum if that is the smallest on study).

The term “salt(s)” as used herein includes acidic and/or basic salts formed with inorganic and/or organic acids and bases. As used herein, the term “pharmaceutically acceptable salt” refers to those salts which are, within the scope of sound medical judgment, suitable for use in contact with the tissues of subjects without undue toxicity, irritation, allergic response and/or the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19.

Pharmaceutically acceptable salts may be formed with inorganic or organic acids. Non-limiting examples of suitable inorganic acids include hydrochloric acid, hydrobromic acid, phosphoric acid, sulfuric acid, and perchloric acid. Non-limiting examples of suitable organic acids include acetic acid, oxalic acid, maleic acid, tartaric acid, citric acid, succinic acid, and malonic acid. Other non-limiting examples of suitable pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. In certain embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, and salicylic acid.

Salts may be prepared in situ during the isolation and purification of the disclosed compound, or separately, such as by reacting the compound with a suitable base or acid, respectively. Non-limiting examples of pharmaceutically acceptable salts derived from bases include alkali metal, alkaline earth metal, ammonium and N⁺(C₁₋₄alkyl)₄ salts. Non-limiting examples of suitable alkali or alkaline earth metal salts include sodium, lithium, potassium, calcium, magnesium, iron, zinc, copper, manganese, and aluminum salts. Further non-limiting examples of suitable pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Non-limiting examples of suitable organic bases from which salts may be derived include primary amines, secondary amines, tertiary amines, substituted amines including naturally occurring substituted amines, cyclic amines, and basic ion exchange resins, such as isopropylamine, trimethylamine, diethylamine, triethylamine, tripropylamine, and ethanolamine. In certain embodiments, pharmaceutically acceptable base addition salts can be chosen from ammonium, potassium, sodium, calcium, and magnesium salts.

As used herein, the term “sensitizing” or equivalents thereof (e.g., “sensitize” or “sensitization”) means making subjects that were previously resistant, non-responsive, or somewhat responsive to a therapy regimen (e.g., chemotherapy, targeted therapy, or immunotherapy) sensitive, responsive, or more responsive to that therapy regimen. In certain embodiments, the term “sensitizing” or equivalents thereof includes “re-sensitizing” or equivalents thereof, making subjects that became resistant, non-responsive, or somewhat responsive to a therapy regimen (e.g., chemotherapy, targeted therapy, or immunotherapy) because of prior exposure to such therapy regimen sensitive, responsive, or more responsive to that therapy regimen.

The term “solvate” represents an aggregate that comprises one or more molecules of a compound of the present disclosure with one or more molecules of a solvent or solvents. Solvates of the compounds of the present disclosure include, for example, hydrates.

The term “subject” generally refers to an organism to which a compound or pharmaceutical composition described herein can be administered. A subject can be a mammal or mammalian cell, including a human or human cell. The term also refers to an organism, which includes a cell or a donor or recipient of such cell. In various embodiments, the term “subject” refers to any animal (e.g., a mammal), including, but not limited to, humans, mammals and non-mammals, such as non-human primates, mice, rabbits, sheep, dogs, cats, horses, cows, chickens, amphibians, reptiles, fish, nematode, and insects, which is to be the recipient of a compound or pharmaceutical composition described herein. Under some circumstances, the terms “subject” and “patient” are used interchangeably herein in reference to a human subject.

The term “synergy,” “synergistic,” “synergistically,” or “enhanced” as used herein refers to an effect of interaction or combination of two or more components to produce a combined effect greater than the sum of their separate effects (or “additive effects”).

As used herein, the terms “treatment,” “treating,” “ameliorating,” and “encouraging” are used interchangeably herein. These terms refer to an approach for obtaining beneficial or desired results including, but not limited to, a therapeutic benefit and/or prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject can still be afflicted with the underlying disorder. For prophylactic benefit, the pharmaceutical composition may be administered to a subject at risk of developing a particular disease, or to a subject reporting one or more of the physiological symptoms of a disease, even though a diagnosis of this disease may not have been made.

The term “treating cancer,” “treatment of cancer,” or an equivalent thereof means to decrease, reduce, or inhibit the replication of cancer cells; decrease, reduce, or inhibit the spread (formation of metastases) of cancer; decrease tumor size; decrease the number of tumors (i.e. reduce tumor burden); lessen or reduce the number of cancerous cells in the body; prevent recurrence of cancer after surgical removal or other anti-cancer therapies; and/or ameliorate or alleviate the symptoms of the disease caused by the cancer.

The at least one cancer sternness inhibitor or the at least one immunotherapeutic agent disclosed herein may be in the form of a pharmaceutical composition. In certain embodiments, the pharmaceutical compositions may comprise at least one cancer sternness inhibitor. In certain embodiments, the pharmaceutical compositions may comprise the at least one compound of formula A and at least one pharmaceutically acceptable carrier. In certain embodiments, the pharmaceutical compositions may comprise at least one immunotherapeutic agent. In certain embodiments, the pharmaceutical compositions may comprise at least one immune checkpoint modulator (e.g., an immune checkpoint inhibitor). In certain embodiments, the pharmaceutical compositions may comprise one or more compounds and at least one pharmaceutically acceptable carrier, where the one or more compounds are capable of being converted into the at least one compound of formula A in a subject (i.e., a prodrug). In certain embodiments, the pharmaceutical compositions may comprise one or more compounds and at least one pharmaceutically acceptable carrier, where the one or more compounds are capable of being converted into the at least one immunotherapeutic agent in a subject (i.e., a prodrug).

The term “carrier” as used herein means a pharmaceutically acceptable material, composition or vehicle, such as, for example, a liquid or solid filler, diluent, excipient, solvent, or encapsulating material, for example, involved in or capable of carrying or transporting the subject pharmaceutical compound from one organ, or portion of the body, to another organ, or portion of the body. Each carrier must be “acceptable” in the sense of being compatible with the other ingredients of the formulation and not injurious to the patient. Non-limiting examples of pharmaceutically acceptable carriers, carriers, and/or diluents include: sugars, such as lactose, glucose and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers, and lubricants, such as sodium lauryl sulfate, magnesium stearate, and polyethylene oxide-polypropylene oxide copolymer as well as coloring agents, release agents, coating agents, sweetening, flavoring and perfuming agents, preservatives, and antioxidants can also be present in the compositions.

Pharmaceutical compositions disclosed herein that are suitable for oral administration may be in the form of capsules, cachets, pills, tablets, lozenges (using a flavored basis, usually sucrose and acacia or tragacanth), powders, granules, a solution in an aqueous or non-aqueous liquid, a suspension in an aqueous or non-aqueous liquid, an oil-in-water emulsion, a water-in-oil emulsion, an elixir, a syrup, pastilles (using an inert base, such as gelatin, glycerin, sucrose, and/or acacia) and/or mouthwashes, each containing a predetermined amount of the at least one compound of the present disclosure.

A pharmaceutical composition disclosed herein may be administered as a bolus, electuary, or paste.

Solid dosage forms for oral administration (capsules, tablets, pills, dragees, powders, granules and the like) may be mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose, and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polypropylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets, and pills, the pharmaceutical compositions may also comprise buffering agents. Solid compositions of a similar type also may be employed as fillers in soft and hard-filled gelatin capsules using such excipients as lactose or milk sugars, as well as high molecular weight polyethylene glycols and the like.

Liquid dosage forms for oral administration may include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, and fatty acid esters of sorbitan, and mixtures thereof. Additionally, cyclodextrins, e.g., hydroxypropyl-β-cyclodextrin, may be used to solubilize compounds.

The pharmaceutical compositions also may include adjuvants such as wetting agents, emulsifying and suspending agents, sweetening, flavoring, coloring, perfuming, and preservative agents. Suspensions, in addition to the compounds according to the disclosure, may contain suspending agents as, such as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Pharmaceutical compositions disclosed herein, for rectal or vaginal administration may be presented as a suppository, which may be prepared by mixing one or more compounds according to the present disclosure with one or more suitable nonirritating excipients or carriers comprising, for example, cocoa butter, polyethylene glycol, a suppository wax or a salicylate, which is solid at room temperature, but liquid at body temperature and, therefore, will melt in the rectum or vaginal cavity and release the compounds of the present disclosure. Pharmaceutical compositions which are suitable for vaginal administration also may include pessaries, tampons, creams, gels, pastes, foams, or spray formulations containing carriers that are known in the art to be appropriate.

Dosage forms for the topical or transdermal administration of a pharmaceutical composition or pharmaceutical tablet of the present disclosure may include powders, sprays, ointments, pastes, creams, lotions, gels, solutions, patches, and inhalants. The pharmaceutical composition or pharmaceutical tablet may be mixed under sterile conditions with a pharmaceutically acceptable carrier, and with any preservatives, buffers, or propellants which may be required.

The ointments, pastes, creams and gels may contain, in addition to the pharmaceutical composition or pharmaceutical tablet of the present disclosure, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays may contain, in addition to a pharmaceutical composition or a pharmaceutical tablet of the present disclosure, excipients such as lactose, talc, silicic acid, aluminum hydroxide, calcium silicates, and polyamide powder, or mixtures of these substances. Additionally, sprays may contain customary propellants, such as chlorofluorohydrocarbons and volatile unsubstituted hydrocarbons, such as butane and propane.

Ophthalmic formulations, eye ointments, powders, solutions, and the like are also contemplated as being within the scope of the present disclosure.

Compositions suitable for parenteral administration may comprise at least one more pharmaceutically acceptable sterile isotonic aqueous or non-aqueous solutions, dispersions, suspensions, emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

The present disclosure reports the surprising discovery that treatment combinations of at least one cancer stemness inhibitor and at least one immunotherapeutic agent have a greater effect in inhibiting cancer cells than the added effects of each of the at least one cancer stemness inhibitor and the at least one immunotherapeutic agent alone.

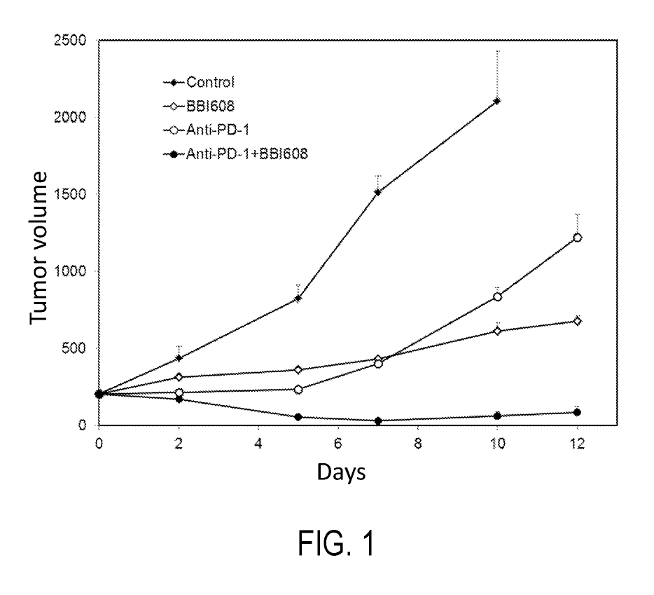

Surprisingly, treatment combinations of at least one cancer stemness inhibitor, for example, BBI608, and at least one immunotherapeutic agent, for example, an anti-PD-1 antibody, resulted in an enhanced anti-tumor effect in a murine CT26 CRC model. CT26 cells share molecular features with aggressive, undifferentiated, refractory human colorectal carcinoma cells. The murine CT26 CRC model is also a microsatellite stable CRC model. As shown in FIG. 1, CT26 tumors displayed an initial response to the anti-PD-1 treatment, but quickly became resistant to the treatment and grew more rapidly after 7 days. BBI608 monotherapy showed lasting anti-tumor activity in the CT26 syngeneic murine CRC model, producing tumor growth inhibition of 76% by the end of treatment. Also as shown in FIG. 1, the treatment combination of BBI608 with an anti-PD-1 antibody produced an enhanced anti-tumor effect, resulting in tumor regression in all treated individuals. Furthermore, 40% of the regressed tumors remained undetectable 30 days after cessation of therapy. No overt toxicity, such as weight loss, unkempt appearance, mortality, and/or other relevant behavior, was observed in any of the groups during the course of the treatment.

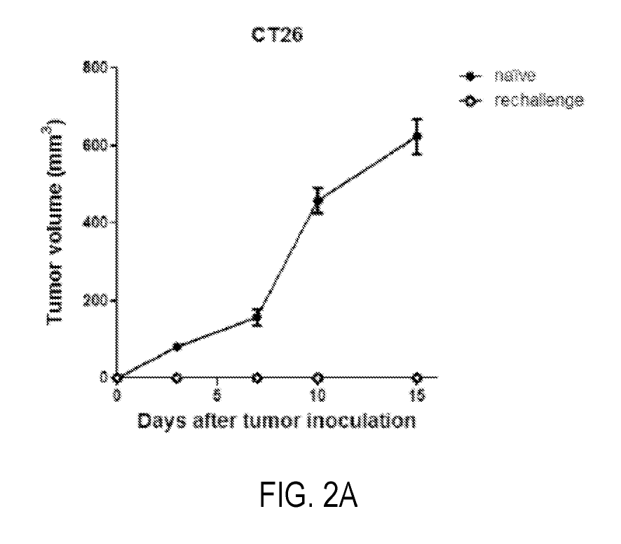

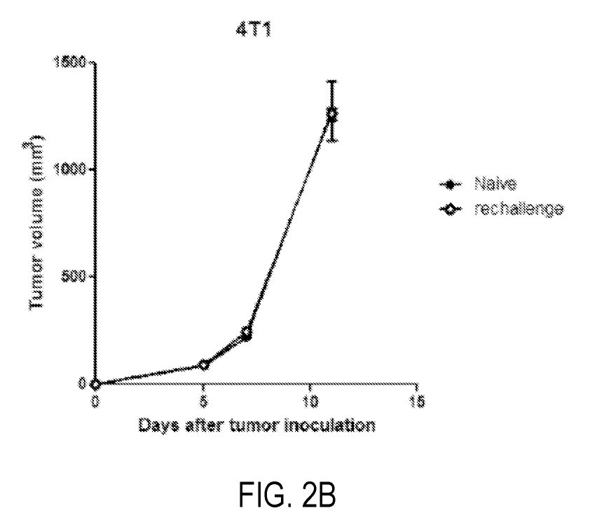

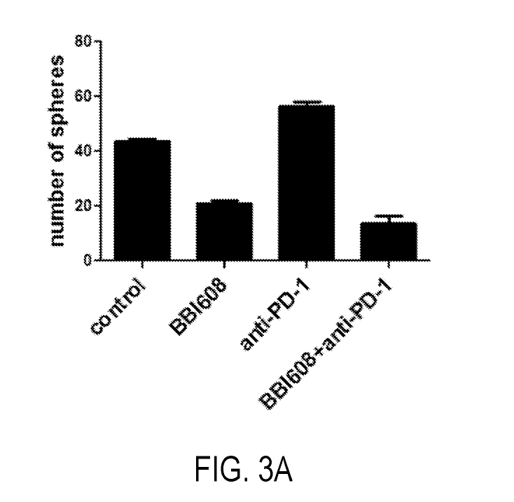

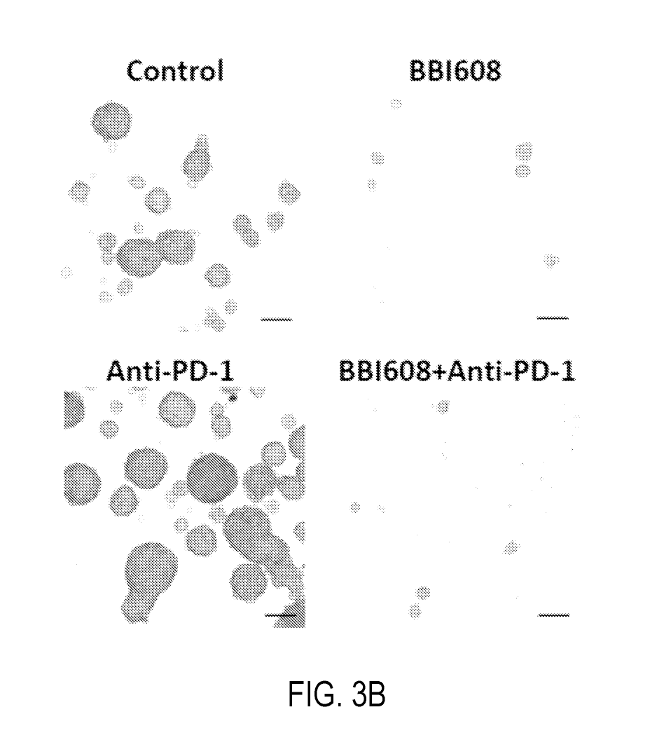

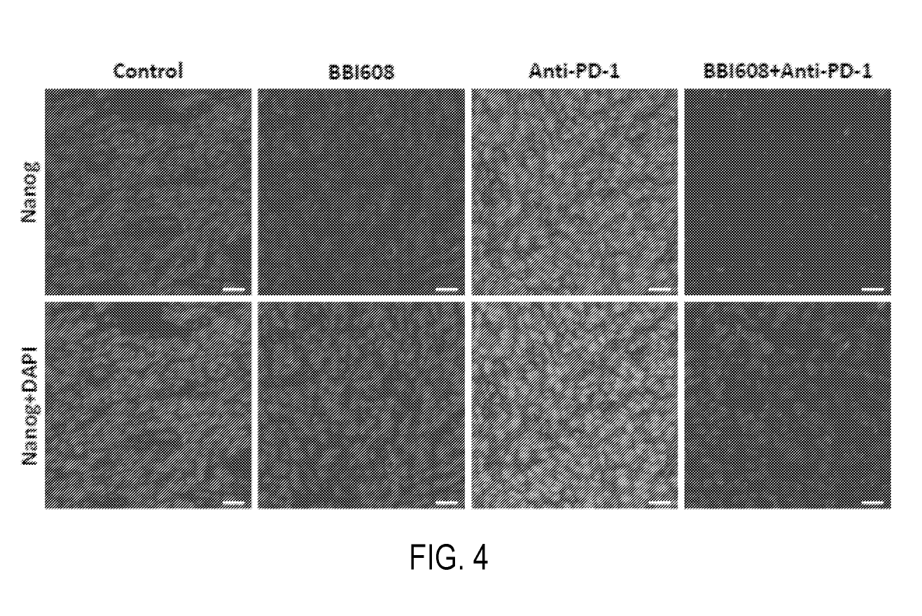

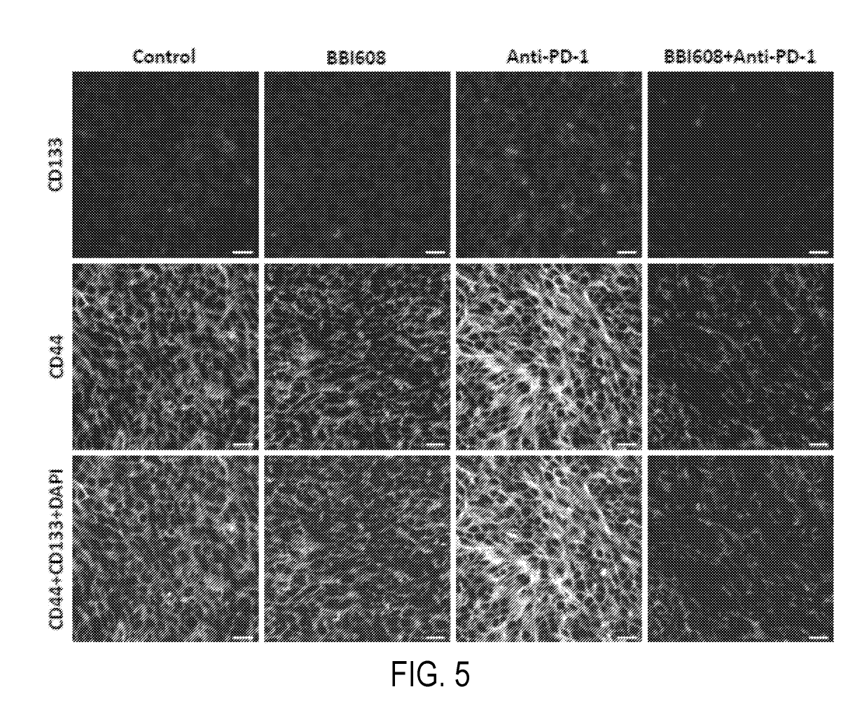

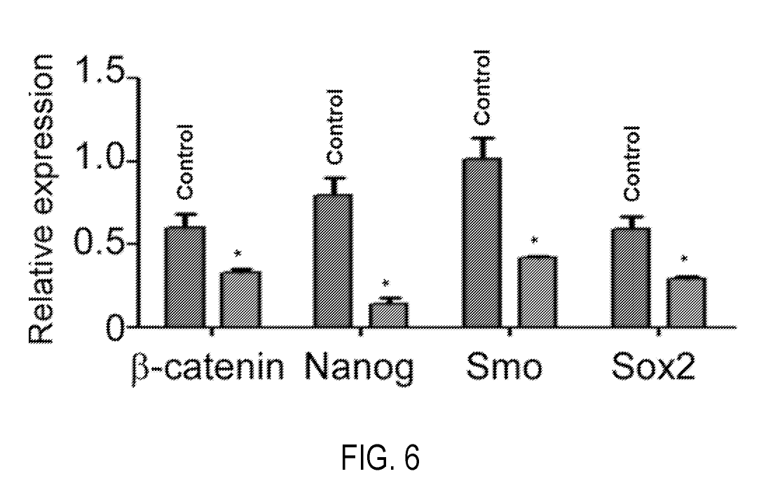

Also surprisingly, the at least one immunotherapeutic agent enhanced cancer stemness and enriched for stemness-high cancer cells. A feature of stemness-high cancer cells is their ability to form tumor spheres under suspension in serum-free medium. In addition, CD133 and CD44 have been wildly used as colorectal cancer stemness markers, and the stem cell factor NANOG plays an important role in the maintenance of stemness properties in stemness-high cancer cells. As shown in FIG. 2 and FIG. 3, tumor cells disassociated from anti-PD-1 antibody treated tumors produced more tumor spheres than untreated control tumor cells. And as shown in FIG. 4 and FIG. 5, control CT26 tumors were found to have moderate levels of NANOG and CD133+ CD44+ cells, but the expression of NANOG, CD44, and CD133 increased significantly in response to anti-PD-1 antibody therapy. As shown in FIGS. 6-9B as well as Table 1, cancer stemness inhibitors were shown to be effective in reducing the levels of NANAOG and CD44+, as well as other markers (e.g., IL-6, CYCLIN D1, MMP-9, BCL2, SMO, SOX2, and β-CATENIN).

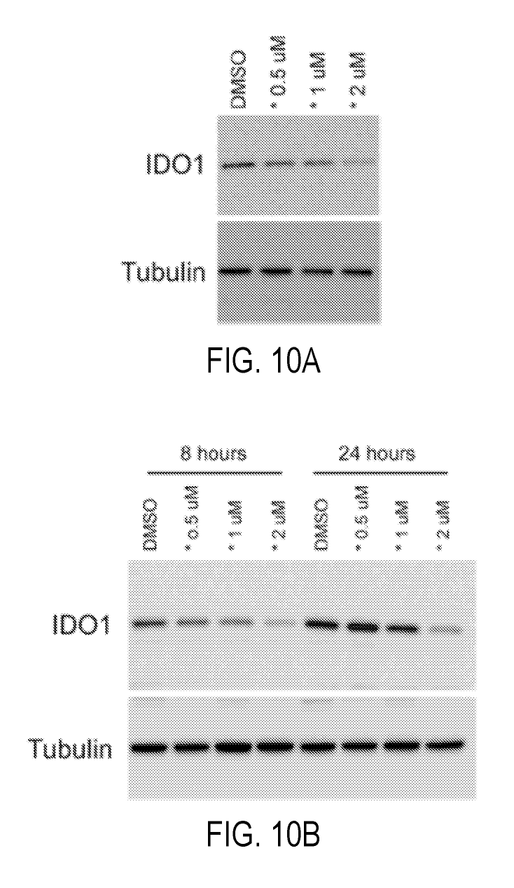

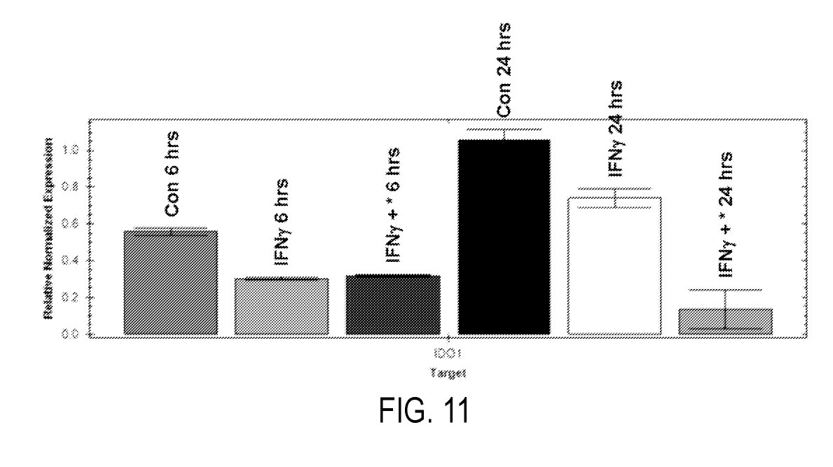

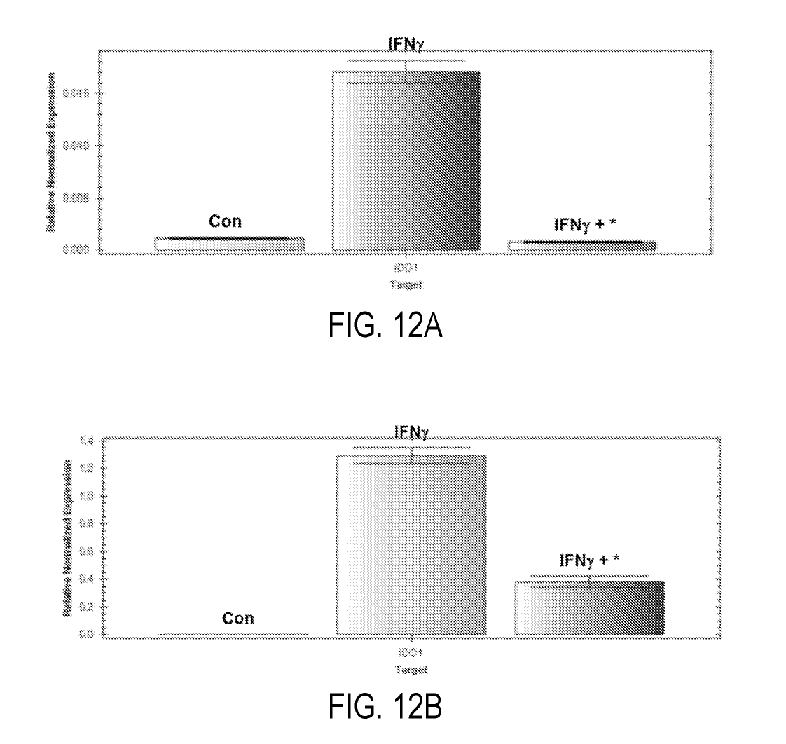

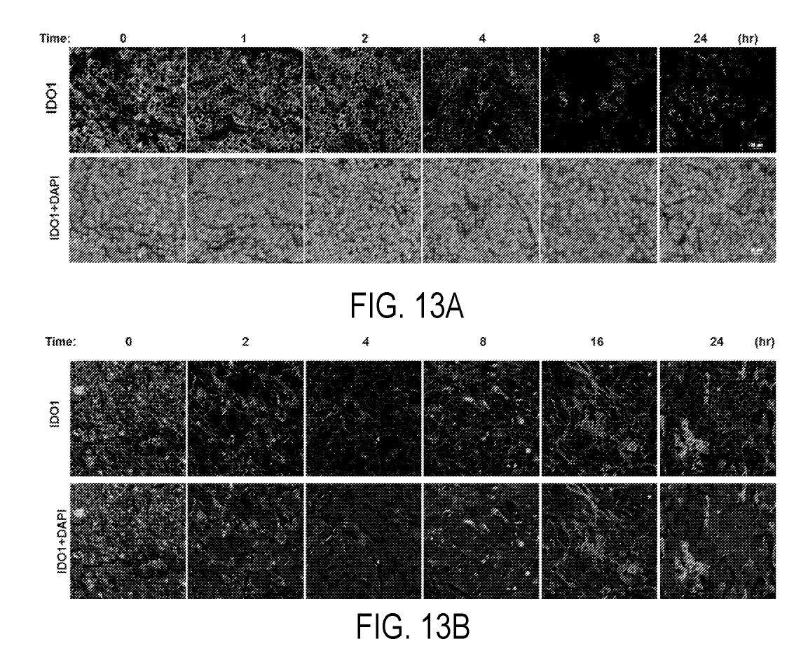

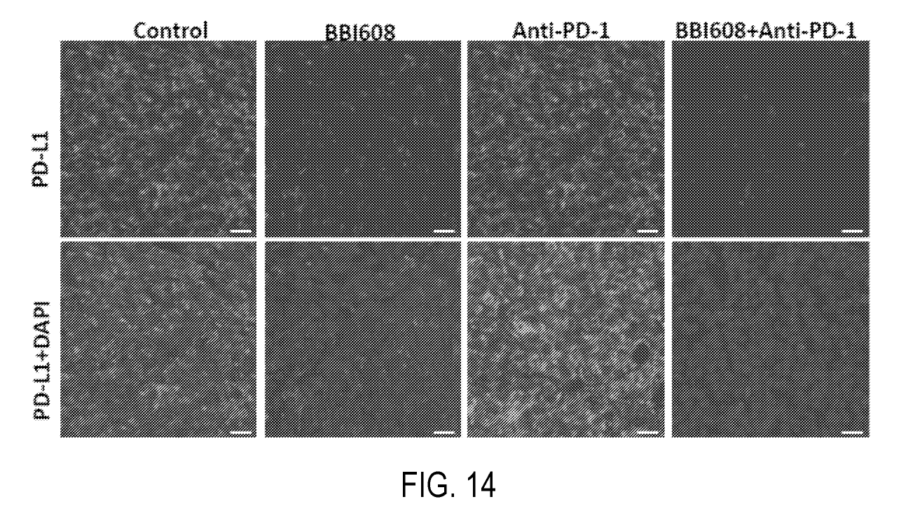

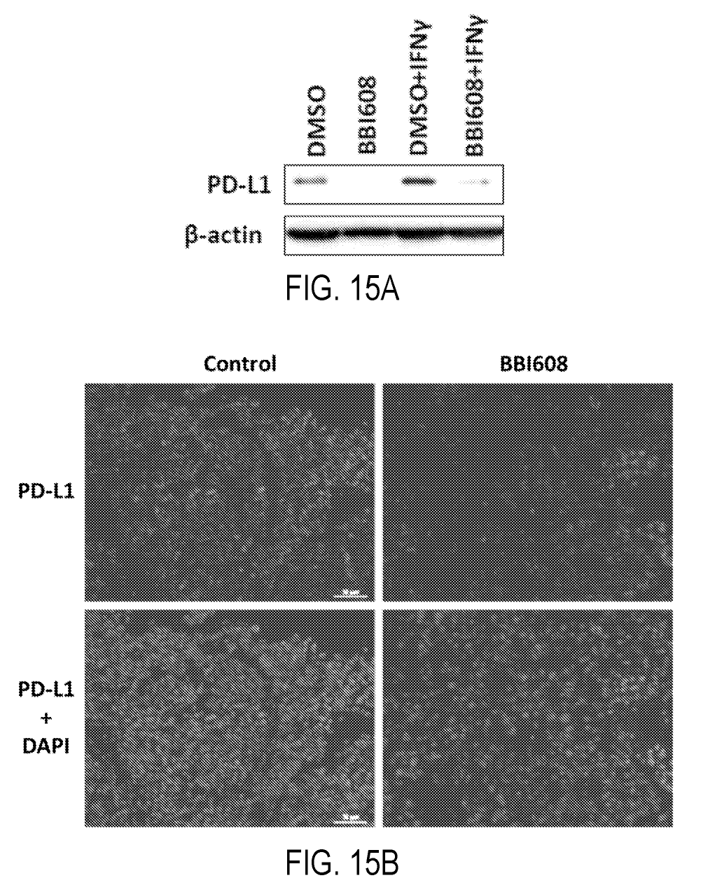

Surprisingly, cancer stemness inhibitors were found to be able to reduce protein expression of at least one immune checkpoint gene. Indoleamine-pyrrole 2,3-dioxygenase-1 (IDO1) and Programmed Death 1 receptor ligand (PD-L1) can inhibit immune checkpoints and assist cancer cells in evading the host immune surveillance. As shown in FIG. 10A and FIG. 10B, the cancer stemness inhibitor BBI608 reduced IDO1 protein levels in both a dose-dependent and a time-dependent manner; and, as shown in FIG. 11, FIG. 12A, and FIG. 12B, BBI608 also inhibited endogenous IDO1 expression and interferon-γ induced IDO1 expression. Time-dependent inhibition of IDO1 expression by cancer stemness inhibitor BBI608 was observed in two different mouse models (see FIG. 13A and FIG. 13B). In addition, as shown in FIG. 14, although PD-L1 expression in CT26 tumor cells was increased by anti-PD-1 antibody treatment, both the BBI608 treatment and the treatment combination of BBI608 and the anti-PD-1 antibody reduced PD-L1 expression. BBI608 further blocked IFN γ-induced PD-L1 overexpression (see FIG. 15A) and down-regulated PD-L1 in vivo (see FIG. 15B).

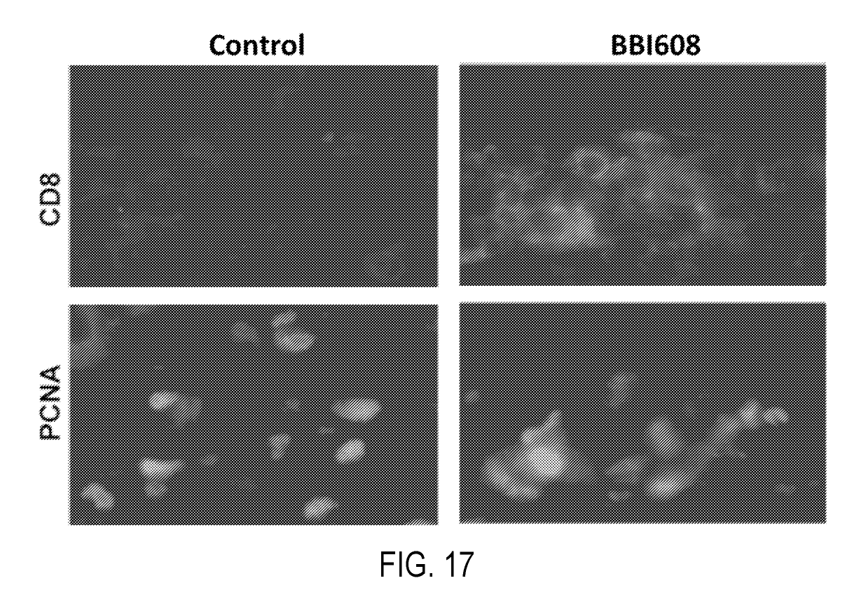

Surprisingly, cancer stemness inhibitors increase T-cell proliferation and activation. In the Apc^{Min/+} mouse model of colon cancer, few CD8⁺ T cells were detected in tumors taken from an untreated control group but, as shown in FIG. 17, treatment with the cancer stemness inhibitor BBI608 led to a significant increase in the number of proliferating tumor infiltrating CD8⁺ T lymphocytes (TILs) present in the tumor.

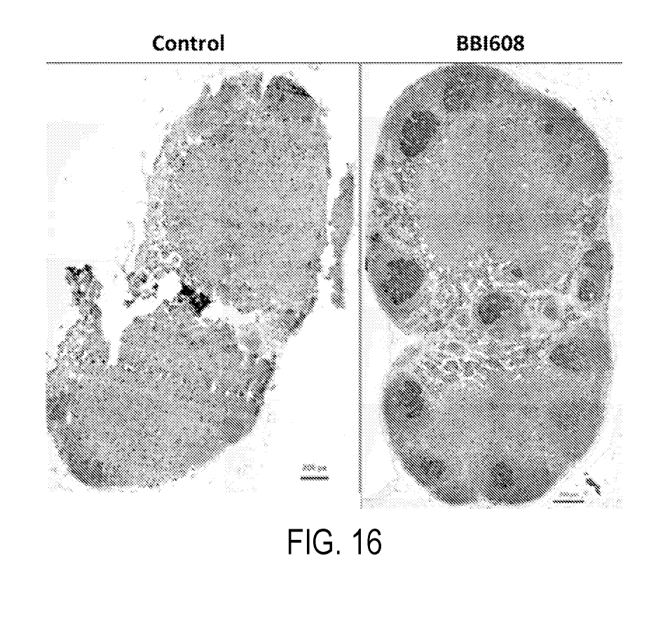

Surprisingly, cancer stemness inhibitors increase other lymphocytes, for example, B-cell, proliferation and activation. For example, as shown in FIG. 16, following treatment of BBI608, multiple centers of B-cell proliferation were observed in the lymph node adjacent to the xenograft B16F10 tumor, indicating that the cancer stemness inhibitor BBI608 induced a B-cell response in vivo.

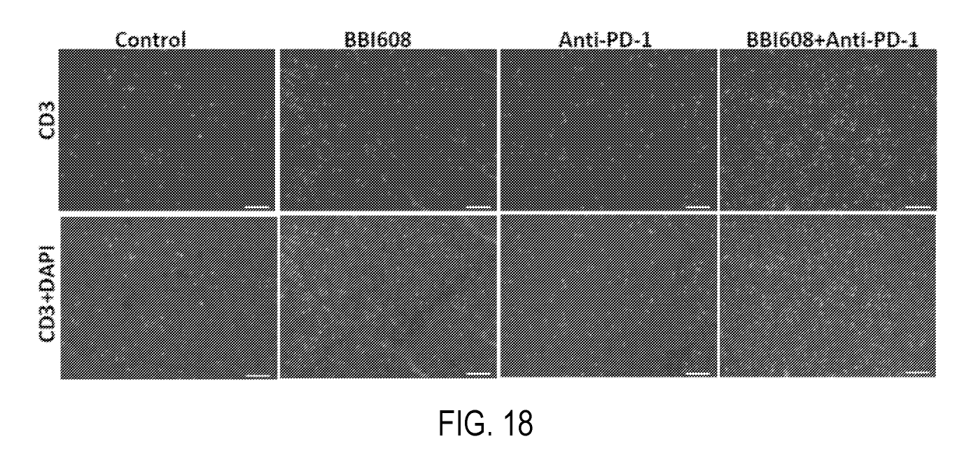

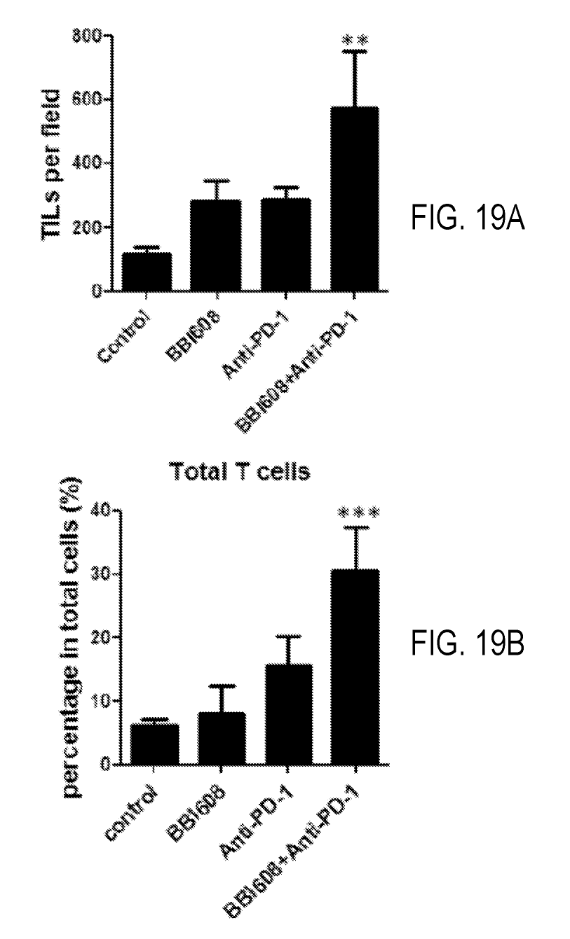

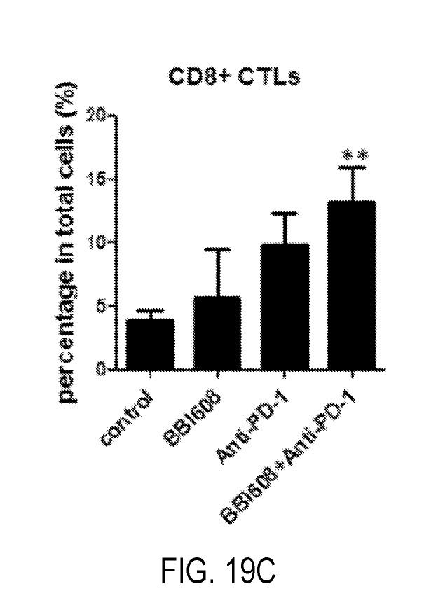

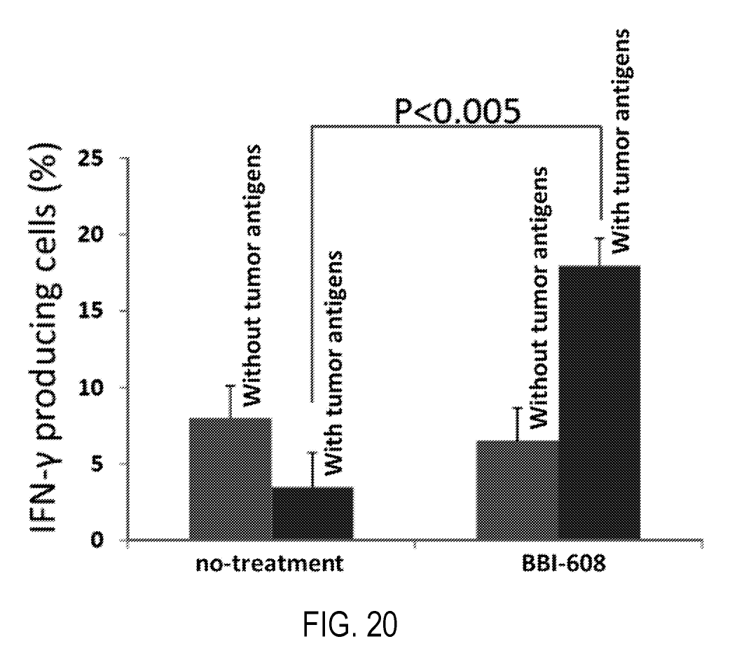

Moreover, as shown in FIG. 18 (by IHC) and FIG. 19 (by FACS), although tumor-infiltrating T lymphocytes (TILs) appeared to increase with BBI608 and anti PD-1 monotherapies, the treatment combination of BBI608 and anti-PD-1 antibody resulted in a more than threefold increase in the number of tumor-infiltrating T cells as compared to untreated control tumors (see FIG. 18 and FIG. 19A). Specifically, the number of tumor infiltrating T cells (CD3⁺) increased more than three fold in the BBI608 and anti-PD-1 treatment combination group over the number of tumor infiltrating T lymphocytes (CD3⁺) detected in tumors taken from control untreated tumors (see FIG. 19A and FIG. 19B) analyzed with two approaches. Similarly, the number of tumor infiltrating cytotoxic T lymphocytes (CD3⁺ and CD8⁺) increased more than twofold in tumors treated with the BBI608 and the anti-PD-1 antibody combination when compared to the number of tumor infiltrating cytotoxic T lymphocytes detected in untreated control tumors (FIG. 19C). Further, as shown in FIG. 20, in the presence of tumor antigen, a higher percentage of CD8⁺ T lymphocytes (cytotoxic T cells) from cancer sternness inhibitor BBI608-treated samples produced INF-γ when compared to CD8⁺ T cells in untreated control samples, indicating that BBI608 increased tumor-specific cytotoxic T lymphocytes proliferation.

Surprisingly, treatment combinations of the present disclosure also resulted in a long-term anti-tumor memory in the treated subjects. As shown in FIG. 21A and FIG. 21B, the BBI608/anti-PD-1 antibody-treated mice that had rejected CT26 tumors and non-treated control mice were inoculated with either with the same CT26 tumor cells or with unrelated murine breast carcinoma 4T1 cells. Unlike non-treated control mice, the BBI608/anti-PD-1 antibody-treated mice were resistant to the CT26 tumor (FIG. 21A) but not to the 4T1 tumor (FIG. 21B). Thus, without being limited to any particular observation or hypothesis, the results suggested that mice cured from CT26 cancer by a treatment combination of a cancer sternness inhibitor and an anti-PD-1 antibody developed a long-term memory to tumor antigens expressed specifically in the treated tumor.

Without being limited to any particular observation or hypothesis, treatment combinations of at least one cancer sternness inhibitor (e.g., BBI608) and at least one immunotherapeutic agent (e.g., an anti-PD-1 antibody) may have a synergistic effect in treating cancer, for example, an effect greater than the additive effects observed after treatment with a cancer sternness inhibitor alone (e.g., BBI608 alone) or an immunotherapeutic agent alone (e.g., an anti-PD-1 antibody alone).

Specifically, the enclosed examples suggest that sternness-high cancer cells are also responsible for anti-PD-1 treatment resistance, for example, in a murine MSS CRC model and cancer sternness-high properties may be responsible for the acquired resistance to anti-PD-1 monotherapy, for example, in the CT26 model. After the CT26 tumors became resistant to anti-PD-1 treatment, the tumors exhibited more of stemness-high phenotype compared to the untreated controls, namely, a higher sphere forming capability in low attachment plates and increased expression of CRC stemness-high markers p-STAT3, NANOG, CD133, and CD44.

Without being limited to any particular observation or hypothesis, it is reasonable to hypothesize that, although the immune evasion mechanisms of stemness-high cancer cells may be multifactorial, the increase p-STAT3 may result in overexpression of PD-L1, which in turn will compete with the administered anti-PD-1 antibody to bind to PD-1 receptor on the surface of T cells and such PD-L1 and PD-1 interaction would inhibit T cells proliferation and survival and likely contribute, at least partially, to the immune resistance of stemness-high cancer cells in CT26 tumors.

Without being limited to a particular observation or hypothesis, the examples discussed herein suggested that treatment combinations of at least one first compound chosen from cancer stemness inhibitors and at least one second compound chosen from immunotherapeutic agents would produce a synergistic effect in inhibiting cancer growth that is greater than the effects of the cancer stemness inhibitor alone or the immunotherapeutic agent alone, or the additive effects of the cancer stemness inhibitor and the immunotherapeutic agent. As shown in the examples, the treatment of stemness-high cancer cells with a cancer stemness inhibitor (e.g., BBI608) led to simultaneous inhibition of stemness-high cancer cell survival and self-renewal and a downregulation of immune checkpoint genes in vitro and in vivo. In addition, the treatment of tumor cells with the combination of a cancer stemness inhibitor and an immunotherapeutic agent (e.g., BBI608/anti-PD-1 antibody) seemed to reduce tumor cells' ability to form spheres in vitro as compared to an untreated control; a cancer stemness inhibitor (e.g., BBI608) seemed to reduce basal and anti-PD-1-induced NANOG, CD44, and CD133 expression, as well as the expression of other cancer stemness markers, including, but not limited to, β-CATENIN, SMO, SOX2, IL-6, CYCLIN D1, MMP-9, and BCL2; a cancer stemness inhibitor (e.g., BBI608) seemed to down-regulate expression of a number of immune checkpoint genes, increased T-cell activation and tumor infiltration, and induced long-term anti-tumor memory; and the combination of a cancer stemness inhibitor and an immunotherapeutic agent (e.g., BBI608/anti-PD-1 antibody) strongly increased CD3⁺ T-cells infiltration inside the tumor, which likely contributed to the rapid regression of tumors after combination therapy was initiated.

In certain embodiments, disclosed herein are methods for treating cancer in a subject comprising administering a therapeutically effective amount of at least one first compound chosen from cancer stemness inhibitors, prodrugs thereof, derivatives thereof, pharmaceutically acceptable salts of any of the foregoing, and solvates of any of the foregoing; and a therapeutically effective amount of at least one second compound chosen from immunotherapeutic agents, prodrugs thereof, derivatives thereof, pharmaceutically acceptable salts of any of the foregoing, and solvates of any of the foregoing.

In certain embodiments, a kit is disclosed that comprises (1) at least one first compound chosen from cancer stemness inhibitors, prodrugs thereof, derivatives thereof, pharmaceutically acceptable salts of any of the foregoing, and solvates of any of the foregoing, and (2) at least one second compound chosen from immunotherapeutic agents, prodrugs thereof, derivatives thereof, pharmaceutically acceptable salts of any of the foregoing, and solvates of any of the foregoing, together with instructions for administration and/or use.

In various embodiments, a composition described herein includes at least one first compound chosen from cancer stemness inhibitors and pharmaceutically acceptable salts thereof, and solvates thereof, and at least one surfactant.

In various embodiments, a composition described herein includes at least one compound chosen from compounds of formula A and pharmaceutically acceptable salts thereof, and solvates thereof, and at least one surfactant.

In certain embodiments, the at least one surfactant is chosen from sodium lauryl sulfate (SLS), sodium dodecyl sulfate (SDS), and polyoxylglycerides. For example, the polyoxylglyceride can be lauroyl polyoxylglycerides (sometimes referred to as Gelucire™) or linoleoyl polyoxylglycerides (sometimes referred to as Labrafil′). Examples of such compositions are disclosed in PCT Patent Application No. PCT/US2014/033566, the content of which is incorporated herein in its entirety.

The present disclosure provides further embodiments of suitable pharmaceutical formulations having selected particle size distribution and methods for identifying an optimum particle size distribution, suitable drug regimen, dosage and interval, suitable methods of preparing 2-acetylnaphtho[2,3-b]furan-4,9-dione including their crystalline forms, and further specific suitable cancer stemness inhibitors as described in the co-owned PCT applications published as WO 2009/036099, WO 2009/036101, WO 2011/116398, WO 2011/116399, and WO 2014/169078, the contents of which are hereby incorporated by reference herein in their entireties.

In certain embodiments, the compounds or pharmaceutical compositions described herein are administered in combination with any of a variety of known therapeutics, including for example, chemotherapeutic and other anti-neoplastic agents, anti-inflammatory compounds, and/or immunosuppressive compounds. In certain embodiments, the compounds, products, and/or pharmaceutical compositions described herein are useful in conjunction with any of a variety of known treatments including, by way of non-limiting example, surgical treatments and methods, radiation therapy, chemotherapy, and/or hormone or other endocrine-related treatment.

In certain embodiments, provided herein is a method of treating cancer in a subject in need thereof, the method comprising administering a therapeutically effective amount of at least one first compound chosen from cancer stemness inhibitors, prodrugs thereof, derivatives thereof, pharmaceutically acceptable salts of any of the foregoing, and solvates of any of the foregoing. In certain embodiments, the method includes administering a therapeutically effective amount of at least one second compound chosen from immunotherapeutic agents, prodrugs thereof, derivatives thereof, pharmaceutically acceptable salts of any of the foregoing, and solvates of any of the foregoing. In certain embodiments, the method comprises administering a treatment combination comprising a therapeutically effective amount of at least one first compound chosen from cancer stemness inhibitors, prodrugs thereof, derivatives thereof, pharmaceutically acceptable salts of any of the foregoing, and solvates of any of the foregoing; and a therapeutically effective amount of at least one second compound chosen from immunotherapeutic agents, prodrugs thereof, derivatives thereof, pharmaceutically acceptable salts of any of the foregoing, and solvates of any of the foregoing. In certain embodiments, the at least one cancer stemness inhibitor is included in a pharmaceutical composition. In certain embodiments, the at least one immunotherapeutic agent is included in a pharmaceutical composition.

In certain embodiments, provided herein is a method of treating a cancer refractory or resistant to an immunotherapeutic agent in a subject in need thereof, the method comprising administering a therapeutically effective amount of at least one first compound chosen from cancer stemness inhibitors, prodrugs thereof, derivatives thereof, pharmaceutically acceptable salts of any of the foregoing, and solvates of any of the foregoing. In certain embodiments, the method includes administering a therapeutically effective amount of at least one second compound chosen from immunotherapeutic agents, prodrugs thereof, derivatives thereof, pharmaceutically acceptable salts of any of the foregoing, and solvates of any of the foregoing. In certain embodiments, the method comprises administering a treatment combination comprising a therapeutically effective amount of at least one first compound chosen from cancer stemness inhibitors, prodrugs thereof, derivatives thereof, pharmaceutically acceptable salts of any of the foregoing, and solvates of any of the foregoing; and a therapeutically effective amount of at least one second compound chosen from immunotherapeutic agents, prodrugs thereof, derivatives thereof, pharmaceutically acceptable salts of any of the foregoing, and solvates of any of the foregoing. In certain embodiments, the at least one cancer stemness inhibitor is included in a pharmaceutical composition. In certain embodiments, the at least one immunotherapeutic agent is included in a pharmaceutical composition.

In certain embodiments, provided herein is a method of preventing cancer relapse in a subject, the method comprising administering a therapeutically effective amount of at least one first compound chosen from cancer stemness inhibitors, prodrugs thereof, derivatives thereof, pharmaceutically acceptable salts of any of the foregoing, and solvates of any of the foregoing. In certain embodiments, the method includes administering a therapeutically effective amount of at least one second compound chosen from immunotherapeutic agents, prodrugs thereof, derivatives thereof, pharmaceutically acceptable salts of any of the foregoing, and solvates of any of the foregoing. In certain embodiments, the method comprises administering a treatment combination comprising a therapeutically effective amount of at least one first compound chosen from cancer stemness inhibitors, prodrugs thereof, derivatives thereof, pharmaceutically acceptable salts of any of the foregoing, and solvates of any of the foregoing; and a therapeutically effective amount of at least one second compound chosen from immunotherapeutic agents, prodrugs thereof, derivatives thereof, pharmaceutically acceptable salts of any of the foregoing, and solvates of any of the foregoing. In certain embodiments, the at least one cancer stemness inhibitor is included in a pharmaceutical composition. In certain embodiments, the at least one immunotherapeutic agent is included in a pharmaceutical composition.

In certain embodiments, provided herein is a method of suppressing regrowth or recurrence of cancer in a subject, the method comprising administering a therapeutically effective amount of at least one first compound chosen from cancer stemness inhibitors, prodrugs thereof, derivatives thereof, pharmaceutically acceptable salts of any of the foregoing, and solvates of any of the foregoing. In certain embodiments, the method includes administering a therapeutically effective amount of at least one second compound chosen from immunotherapeutic agents, prodrugs thereof, derivatives thereof, pharmaceutically acceptable salts of any of the foregoing, and solvates of any of the foregoing. In certain embodiments, the method comprises administering a treatment combination comprising a therapeutically effective amount of at least one first compound chosen from cancer stemness inhibitors, prodrugs thereof, derivatives thereof, pharmaceutically acceptable salts of any of the foregoing, and solvates of any of the foregoing; and a therapeutically effective amount of at least one second compound chosen from immunotherapeutic agents, prodrugs thereof, derivatives thereof, pharmaceutically acceptable salts of any of the foregoing, and solvates of any of the foregoing. In certain embodiments, the at least one cancer stemness inhibitor is included in a pharmaceutical composition. In certain embodiments, the at least one immunotherapeutic agent is included in a pharmaceutical composition.

In certain embodiments, provided herein is a method of treating cancer in a subject, the method comprising measuring an expression level of an immune checkpoint gene in a biological sample obtained from a subject diagnosed of a cancer; confirming that the expression level of the immune checkpoint gene is above a benchmark level; and administering to the subject a therapeutically effective amount of at least one first compound chosen from cancer stemness inhibitors, prodrugs thereof, derivatives thereof, pharmaceutically acceptable salts of any of the foregoing, and solvates of any of the foregoing. In certain embodiments, the immune checkpoint gene expresses a biomarker chosen from PD-1, PD-L1, PD-L2, CTLA-4, IDO1, STAT3, IL-6, or other immune checkpoint proteins. In certain embodiments, the immune check point gene is related to PD-L1, PD-L2, IDO1, or IL6. In certain embodiments, the immune check point gene is related to PD-L1, PD-L2, or IDO1.

In certain embodiments, the method comprises measuring an expression level of a cancer stemness gene in a biological sample obtained from a subject diagnosed of a cancer; and confirming that the expression level of the cancer stemness gene is above a benchmark level. In certain embodiments, the cancer stemness gene expresses a biomarker chosen from β-CATENIN, NANOG, SMO, SOX2, STAT3, AXL, ATM, c-MYC, KLF4, SURVIVIN, or BMI-1. In certain embodiments, the cancer stemness gene expresses a biomarker chosen from β-CATENIN, NANOG, SMO, SOX2, or c-MYC.

In certain embodiments, the method includes administering a therapeutically effective amount of at least one second compound chosen from immunotherapeutic agents, prodrugs thereof, derivatives thereof, pharmaceutically acceptable salts of any of the foregoing, and solvates of any of the foregoing.

In certain embodiments, the method comprises administering a treatment combination comprising a therapeutically effective amount of at least one first compound chosen from cancer stemness inhibitors, prodrugs thereof, derivatives thereof, pharmaceutically acceptable salts of any of the foregoing, and solvates of any of the foregoing; and a therapeutically effective amount of at least one second compound chosen from immunotherapeutic agents, prodrugs thereof, derivatives thereof, pharmaceutically acceptable salts of any of the foregoing, and solvates of any of the foregoing. In certain embodiments, the at least one cancer stemness inhibitor is included in a pharmaceutical composition. In certain embodiments, the at least one immunotherapeutic agent is included in a pharmaceutical composition.

In certain embodiments, provided herein is a method of treating cancer in a subject comprising administering to a subject a therapeutically effective amount of at least one first compound chosen from cancer stemness inhibitors, prodrugs thereof, derivatives thereof, pharmaceutically acceptable salts of any of the foregoing, and solvates of any of the foregoing, where the subject has an immune checkpoint gene expression level above a benchmark level. In certain embodiments, the cancer is refractory or resistant to an immunotherapeutic agent.

In certain embodiments, provided herein is a method of sensitizing or re-sensitizing cancer cells to an immunotherapeutic agent, the method comprising administering to cancer cells at least one first compound chosen from cancer stemness inhibitors, prodrugs thereof, derivatives thereof, pharmaceutically acceptable salts of any of the foregoing, and solvates of any of the foregoing, where the subject has an immune checkpoint gene expression level above a benchmark level. In certain embodiments, the cancer cells are in a subject. In certain embodiments, the immune check point gene expresses at least one biomarker chosen from PD-L1, PD-L2, IDO1, or/and IL6, or proteins that suppress immune response.

In certain embodiments, the subject has a cancer stemness gene expression level above a benchmark level. In certain embodiments, the cancer stemness gene expresses at least one biomarker chosen from β-CATENIN, NANOG, SMO, SOX2, STAT3, AXL, ATM, c-MYC, KLF4, SURVIVIN, or BMI-1.

In certain embodiments, a subject's expression levels of a cancer stemness gene or an immune checkpoint gene is considered to be above respective benchmark levels if more than, e.g., 10% tumor cells express, e.g., IDO1, or if the cancer is associated with β-CATENIN localization in cell nucleus as opposed to cell membrane. Accordingly, in certain embodiments, the method includes detecting a locus of β-CATENIN expression in a patient's tissue sample, where the locus of such β-CATENIN expression is used as a biomarker for patient selection. In certain embodiments, significant β-CATENIN expression is detected in the cell nucleus. In certain embodiments, the medium to strong expression of β-CATENIN is detected in, e.g., 20% or more tumor cells.

In certain embodiments, the method includes administering a therapeutically effective amount of at least one second compound chosen from immunotherapeutic agents, prodrugs thereof, derivatives thereof, pharmaceutically acceptable salts of any of the foregoing, and solvates of any of the foregoing. In certain embodiments, the method comprises administering a treatment combination comprising a therapeutically effective amount of at least one first compound chosen from cancer stemness inhibitors, prodrugs thereof, derivatives thereof, pharmaceutically acceptable salts of any of the foregoing, and solvates of any of the foregoing; and a therapeutically effective amount of at least one second compound chosen from immunotherapeutic agents, prodrugs thereof, derivatives thereof, pharmaceutically acceptable salts of any of the foregoing, and solvates of any of the foregoing. In certain embodiments, the at least one cancer stemness inhibitor is included in a pharmaceutical composition. In certain embodiments, the at least one immunotherapeutic agent is included in a pharmaceutical composition.

In certain embodiments, provided herein is a method of determining suitable benchmark expression levels of cancer stemness genes or/and immune checkpoint genes. In certain embodiments, provided herein are methods of screening subjects by using putative biomarkers. In certain embodiments, provided herein is a method of treating cancer in a subject comprising providing pharmaceutical formulations having selected particle size distribution. In certain embodiments, provided herein is a method identifying an optimum particle size distribution, suitable drug regimen, or dosage and interval. In certain embodiments, provided herein are methods of preparing 2-acetylnaphtho[2,3-b]furan-4,9-dione including their crystalline forms. Some of the methods are described in PCT applications published as WO 2009/036099, WO 2009/036101, WO 2011/116398, WO 2011/116399, and WO 2014/169078, the contents of which are incorporated herein in their entirety by reference.

In certain embodiments, provided herein is a method of sensitizing or re-sensitizing cancer cells to an immunotherapeutic agent, the method comprising administering to cancer cells at least one first compound chosen from cancer stemness inhibitors, prodrugs thereof, derivatives thereof, pharmaceutically acceptable salts of any of the foregoing, and solvates of any of the foregoing. In certain embodiments, the method sensitizes or re-sensitizes cancer cells to at least one immune response. In certain embodiments, the cancer cells are in a subject. In certain embodiments, the method comprises administering a treatment combination comprising a therapeutically effective amount of at least one first compound chosen from cancer stemness inhibitors, prodrugs thereof, derivatives thereof, pharmaceutically acceptable salts of any of the foregoing, and solvates of any of the foregoing; and a therapeutically effective amount of at least one second compound chosen from immunotherapeutic agents, prodrugs thereof, derivatives thereof, pharmaceutically acceptable salts of any of the foregoing, and solvates of any of the foregoing. In certain embodiments, the at least one cancer stemness inhibitor is included in a pharmaceutical composition. In certain embodiments, the at least one immunotherapeutic agent is included in a pharmaceutical composition.

In certain embodiments, the method comprises sensitizing or re-sensitizing cancer cells to an immunotherapeutic agent by a plurality of methods. In certain embodiments, the method comprises changing the level of one or more proteins that are capable of assisting cancer cells to escape from the immune system. In certain embodiments, the method comprises changing the expression of an immune checkpoint gene. In certain embodiments, the method comprises reducing the expression of an immune check point gene. In certain embodiments, the method comprises changing (for example, reducing) the immune suppression caused by cancer cells. In certain embodiments, the method comprises changing the microenvironment of tumor cells. In certain embodiments, the method comprises reducing the levels of one or more ligands to programmed cell death protein 1 (PD1). In certain embodiments, the method comprises reducing the level of PD-L1 or/and PD-L2. In certain embodiments, the method comprises reducing the level of indoleamine 2,3-dioxygenase (IDO-1). In certain embodiments, the method comprises reducing the level of T cell Ig- and mucin-domain-containing molecule-3 (TIM-3). In certain embodiments, the method comprises reducing the level of prostaglandin E2 (PGE2).

In certain embodiments, provided herein is a method of increasing the number of immune cells, increasing the survival of immune cells, or activating immune cells in or around cancer cells, the method comprising administering to cancer cells at least one first compound chosen from cancer stemness inhibitors, prodrugs thereof, derivatives thereof, pharmaceutically acceptable salts of any of the foregoing, and solvates of any of the foregoing. In certain embodiments, the method comprises increasing the presence and/or activity of one or more immune cells. In certain embodiments, the method comprises increasing the level of the immune cells. In certain embodiments, the method comprises increasing the survival of the immune cells. In certain embodiments, the method comprises activating the immune cells. For example, the immune cells can include leukocytes. Examples of leukocytes can include lymphocytes (including T cells, T helper cells, and natural killer cells) or/and antigen presenting cells (including dendritic cells). In certain embodiments, the method comprises increasing the infiltration of T cells (for example, cytotoxic T cells or CD8⁺ cells) into cancer cells. In certain embodiments, the method comprises increasing the survival of T cells (for example, cytotoxic T cells or CD8⁺ cells) in or around cancer cells. In certain embodiments, the method comprises increasing the recruitment of antigen presenting cells (for example, dendritic cells) in or around cancer cells. In certain embodiments, the method comprises increasing the level of major histocompatibility complex (MEW) class II molecules. In certain embodiments, the method comprises increasing the level of interleukin-10 (IL-10). In certain embodiments, the cancer cells are in a subject. In certain embodiments, the method comprises administering a treatment combination comprising a therapeutically effective amount of at least one first compound chosen from cancer stemness inhibitors, prodrugs thereof, derivatives thereof, pharmaceutically acceptable salts of any of the foregoing, and solvates of any of the foregoing; and a therapeutically effective amount of at least one second compound chosen from immunotherapeutic agents, prodrugs thereof, derivatives thereof, pharmaceutically acceptable salts of any of the foregoing, and solvates of any of the foregoing. In certain embodiments, the at least one cancer stemness inhibitor is included in a pharmaceutical composition. In certain embodiments, the at least one immunotherapeutic agent is included in a pharmaceutical composition.

In certain embodiments, the cancer is chosen from esophageal cancer, gastroesophageal junction cancer, renal cell carcinoma, lung cancer, gastrointestinal cancer, leukemia, lymphoma, myeloma, brain cancer, pancreatic cancer, endometrial cancer, prostate cancer, liver cancer, bladder cancer, gastroesophageal adenocarcinoma, chondrosarcoma, colorectal adenocarcinoma, microsatellite instability-high metastatic colorectal cancer, microsatellite stable metastatic colorectal cancer, colorectal cancer with mismatch-repair deficiency, colorectal cancer without mismatch-repair deficiency, breast cancer, renal cell carcinoma, ovarian cancer, head and neck cancer, melanoma, gastric adenocarcinoma, sarcoma, genitourinary cancer, gynecologic cancer, or adrenocorticoid carcinoma. In certain embodiments, the cancer is melanoma. In certain embodiments, the cancer is breast cancer. In certain embodiments, the cancer is bladder cancer. In certain embodiments, the cancer is renal cell carcinoma. In certain embodiments, the cancer is colorectal cancer. In certain embodiments, the cancer is colorectal adenocarcinoma. In certain embodiments, the cancer is microsatellite instability-high metastatic colorectal cancer. In certain embodiments, the cancer is microsatellite stable metastatic colorectal cancer. In certain embodiments, the cancer is colorectal cancer with mismatch-repair deficiency. In certain embodiments, the cancer is colorectal cancer without mismatch-repair deficiency. In certain embodiments, the cancer is pancreatic cancer. In certain embodiments, the cancer is endometrial cancer.

In certain embodiments, the cancer may be unresectable. In certain embodiments, the cancer may be advanced. In certain embodiments, the cancer may be refractory. In certain embodiments, the cancer may be recurrent. In certain embodiments, the cancer may be metastatic.

In certain embodiments, provided herein is a kit comprising (1) at least one first compound chosen from cancer stemness inhibitors, prodrugs thereof, derivatives thereof, pharmaceutically acceptable salts of any of the foregoing, and solvates of any of the foregoing, and (2) at least one second compound chosen from immunotherapeutic agents, prodrugs thereof, derivatives thereof, pharmaceutically acceptable salts of any of the foregoing, and solvates of any of the foregoing, together with instructions for administration and/or use.

### EXAMPLES

Examples are provided below to further illustrate different features of the present invention. The examples also illustrate useful methodology for practicing the invention. These examples do not limit the claimed invention.

### Example 1: Treatment of CT26 Murine Colon Carcinoma Xenograft with BBI-608, and/or an Anti-PD1 Antibody

### CT26 Murine Colon Carcinoma Xenograft Model

All BALB/c mice (Taconic, Hudson, N.Y., USA) were housed in Association for Assessment and Accreditation of Laboratory Animal Care approved facilities in static microisolator cages. Murine MSS-status colon carcinoma CT26 cells (ATCC CRL-2639) and murine breast carcinoma cells 4T1 (ATCC CRL-2539) were purchased from American Type Culture Collection (ATCC, Manassas, Va., USA), and grown in RPMI-1640 medium (ATCC) supplemented with 10% heat-inactivated fetal calf serum. After harvesting during exponential growth, tumors were initiated by subcutaneously implanting 3×10⁵ CT26 tumor cells into the right dorsal flank of each 8-12 week old female BALB/c mouse. When the tumor volume reached about 200 mm³, the mice were randomized into four groups and treated with either rat immunoglobulin (Ig) G (Sigma-Aldrich, St. Louis, Mo., USA) at 10 mg/kg (iv. q4d) as control, BBI608 at 100 mg/kg (po. qd) by oral gavage, anti-PD-1 antibody at 10 mg/kg (BioXcell, West Lebanon, N.H., USA, clone RMP1-14, iv. q4d), or both BBI608 at 100 mg/kg (po. qd) by oral gavage and anti-PD-1 antibody at 10 mg/kg (BioXcell, West Lebanon, N.H., USA, clone RMP1-14, iv. q4d) for 11 consecutive days (n=5/group). Body weight and clinical signs were monitored throughout the course of the treatment in accordance with the Institutional Animal Care and Use Committee approved protocols. Drug efficacy was analyzed by measuring tumor volume in mm³ calculated by multiplying 0.5×width²×length. Representative results are presented for experiments which were repeated at least three times.

### Results

As shown in FIG. 1, CT26 tumors only displayed an initial response to anti-PD-1 treatment, and quickly became resistant and grew more rapidly after 7 days on treatment. BBI608 monotherapy showed a lasting anti-tumor activity in the CT26 syngeneic murine CRC model, producing tumor growth inhibition of 76% by the end of treatment. Conversely, the combined treatment of BBI608 with the anti-PD-1 antibody produced a synergistic antitumor effect, resulting in tumor regression in all treated individuals (FIG. 1). Furthermore, 40% of the regressed tumors remained undetectable 30 days after cessation of therapy. No overt toxicity, as defined by weight loss, unkempt appearance, mortality, and behavior, was observed in any of the groups during the course of the treatment.

### Example 2: Tumor Re-Challenge After Treatment With BBI-608, and/or an Anti-PD1 Antibody

Thirty days after the initiation of treatment with BBI-608 and the anti-PD-1 antibody, 10 mice that showed complete tumor rejection were re-challenged with tumor cells. Five BBI608/anti-PD1 antibody treated mice that rejected the CT26 tumor cell xenograft were injected again with either 3×10⁵ CT26 or 3×10⁵ 4 T1 cells into the left dorsal flank. As a control, either 3×10⁵ CT26 or 3×10⁵ 4 T1 cells were injected into the left dorsal flank of five naïve, non-treated mice.

As shown in FIG. 2A and FIG. 2B, mice which had rejected CT26 tumors were challenged either with the same CT26 tumor cells or with unrelated murine breast carcinoma 4T1 cells. Compared with naïve mice inoculated with the same cells, the rechallenged mice were resistant to the CT26 tumor but not to the 4T1 tumor. This result indicates that the mice cured by the BBI608 and anti-PD-1 antibody combination therapy had long-term memory to tumor antigens expressed specifically in the CT26 tumor.

### Example 3: Formation of Tumor Spheres After Treatment With BBI-608, and/or an Anti-PD1 Antibody

Portions of tumor tissues were dissociated into a single cell suspension by enzymatic digestion with DMEM (Gibco) containing 200 U/mL Collagenase (Sigma) and 100 U/mL DNAse I (Sigma) at 37° C. for 30 minutes. Cells were then filtered through 40 μm strainers and incubated for 5 min at room temperature in ACK lysis buffer (Thermo Fisher) to remove red blood cells. 1000 live tumor cells, as assessed by Trypan blue (Gibco) staining, were then suspended in 1 mL sphere medium and plated on a low-attachment cell culture 12-well plate in triplicate. Cancer sphere culture medium included B-27 (Gibco), 20 ng/ml EGF (R&D), 10 ng/ml basicFGF (R&D), 0.4% BSA Gemini, and 0.3% agarose in DMEM/F12 (Gibco). After 10 days in culture, the number of tumor spheres was counted.

Most of the tumor cells in the CT26 tumor control group had low levels of active p-STAT3, only a small portion of tumor cells had strong p-STAT3 staining. After anti-PD-1 antibody treatment, the intensity of p-STAT3 was increased. BBI608 reduced p-STAT3 levels both in the BBI608 single therapy group and in the BBI608 and anti-PD-1 antibody combination groups.

As shown in FIG. 3A and FIG. 3B, tumor cells disassociated from anti-PD-1 antibody treated tumors produced more tumor spheres than control, while BBI608 alone and the BBI608/anti-PD-1 antibody combination therapy groups both had significantly lower numbers of spheres than control.

### Example 4: Analysis of Gene Expression and Cell Surface Markers After Treatment With BBI-608 and/or an Anti-PD1 Antibody

### Analysis of Gene Expression by Immunofluorescence

At the end of treatment, tumors were harvested from euthanized mice. Part of the dissected tumors were fixed overnight in 3.7% or 10% neutral buffered formaldehyde at 4° C., and then paraffin embedded, cut to 4-5 micron sections, and affixed onto positively charged slides. After baking and deparaffinization, the slides with tumor or control tissues were incubated in a 10 mM sodium citrate solution pH=6.0 for antigen retrieval at 98° C. Afterwards, slides were probed with primary antibodies against P-STAT3 (Tyr705) (rabbit, Cell Signaling, 1:100), β-CATENIN (mouse, Santa Cruz, 1:400), IL-6 (mouse, Novus Biol., 1:100), PD-L1 (rabbit, Cell Signaling, 1:100), PCNA (mouse, Santa Cruz, 1:5000), CD8a (rabbit, Santa Cruz, 1:30), CD44 (rat, BioLegend, 1:50), CD44 (mouse, Cell Signaling, 1:100), CD133 (mouse, Miltenyi, 1:100), IDO1 (mouse, Millipore 1:100), or/and CD3 (rabbit, Abcam, 1:100) at 4° C. overnight, and then AlexaFluor fluorescent dye-conjugated secondary antibodies (Invitrogen, 1:300 or 1:500) at room temperature for one hour. After mounting in ProLong mounting medium containing DAPI (Invitrogen), the slides were examined on a Zeiss Axio Imager M2 upright fluorescence microscope with a 20× objective and analyzed with Zen software.

### Analysis of Gene Expression by Western-Blotting

3×10⁵ CT26 cells in 6-well plated were treated with 100 ng/ml IFNγ for 24 hours at the presence of control DMSO or 1 μM BBI608. Cells were washed twice with ice-cold PBS and lysed in lysis buffer [50 mM Hepes (pH 7.5), 1% Nonidet P-40, 150 mM NaCl, 1 mM EDTA, and 1× protease and phosphatase inhibitor mixture (EMD Millipore)]. Soluble protein (20 μg) was separated by SDS/PAGE and transferred to nitrocellulose membranes. Primary antibodies against P-STAT3 (Y705), PD-L1, and ACTIN (Sigma) were used in this study. The antigen-antibody complexes were visualized by enhanced chemiluminescence (BioRad).

### Analysis of Cell Surface Marker Expression by FACS Analysis

Tumors were dissociated to a single cell suspension as described above. Following ACK lysis, cells were counted and suspended in PBS at a concentration of 10⁶/100 μL. Dead cells were then labeled with Zombie NIR dye (Invitrogen) and after Fc blocking, the cells were incubated with antibodies purchased from BioLegend including the following: CD3 (clone 17A2), CD4 (clone RM4-5), and CD8a (clone 53-6.7). The stained cells were then analyzed using a BD LSRFortessa. Cells negative for Zombie NIR dye were further analyzed for T cell surface marker staining.

### Statistical Analysis of Gene Expression

Results are presented as mean±standard error. Statistical significance amongst test groups was determined with a 1 way ANOVA using GraphPad Prism V5.00 and an alpha of 0.05. A post hoc analysis using the Tukey method was performed to test significance between groups and a p value <0.05 was considered significant.

### Results

The changes in gene expression following treatment with BBI608 were analyzed. FaDu sphere cultures were treated for 6 hours with DMSO (control) or BBI608 at 2 mM. Total RNA was isolated, reversed transcribed and the generated cDNA was analyzed using a qPCR cancer stem cell array. Data was normalized to the expression of the house keeping gene, GAPDH. The normalized expression of numerous key molecular markers and genes responsible for cancer stem cell proliferation and self-renewal, among them, e.g., NANOG, AXL, ATM, STAT3, and BMI-1, were found to be downregulated by treatment with BBI608, as shown in Table 1 below.

TABLE 1

| Gene | % Change |
|---|---|
| NANOG | −93.34 |
| KLF17 | −90.18 |
| CD34 | −88.18 |
| LIN28A | −87.52 |
| POU5F1 | −77.31 |
| PECAM1 | −70.26 |
| ATM | −68.65 |
| MERTK | −65.78 |
| NOTCH2 | −64.37 |
| LATS1 | −60.75 |
| ITGA2 | −60.71 |
| SMO | −55.56 |
| TGFBR1 | −53.34 |
| MAML1 | −52.82 |
| WWC1 | −51.64 |
| ITGA6 | −54.63 |
| ITGB1 | −50.45 |
| AXL | −48.50 |
| KITLG | −47.99 |
| JAK2 | −47.94 |
| YAP1 | −47.45 |
| BMI1 | −47.40 |
| NOTCH1 | −46.24 |
| ATXN1 | −45.26 |
| ERBB2 | −43.81 |
| SIRT1 | −43.35 |
| WEE1 | −42.86 |
| FGFR2 | −41.18 |
| DDR1 | −38.59 |
| GSK3B | −38.06 |
| ENG | −37.62 |
| DACH1 | −36.55 |
| ALCAM | −36.13 |
| HDAC1 | −36.10 |
| CD44 | −35.44 |
| HPRT1 | −33.81 |
| IKBKB | −32.11 |
| DNMT1 | −32.09 |
| ETFA | −31.12 |
| FOXP1 | −29.76 |
| FLOT2 | −29.19 |
| CHEK1 | −28.88 |
| B2M | −27.69 |
| MUC1 | −27.42 |
| CD24 | −26.46 |
| NFKB1 | −25.54 |
| ACTB | −24.49 |
| EPCAM | −22.87 |
| STAT3 | −22.87 |
| TWIST2 | −21.59 |
| PLAUR | −20.64 |
| EGF | −19.07 |
| ALDH1A1 | −18.18 |
| RPLP0 | −17.48 |
| TAZ | −15.09 |
| JAG1 | −14.24 |
| ID1 | −12.68 |
| ITGA4 | −11.52 |
| IL8 | −7.62 |
| MYC | −3.91 |

As shown in FIG. 4 and FIG. 5, control CT26 tumors were found to have moderate levels of NANOG, as well as CD133⁺ CD44⁺ cells. Anti-PD-1 antibody therapy increased NANOG, CD44, and CD133 expression, whereas BBI608 reduced basal and anti-PD-1 antibody-induced NANOG, CD44, and CD133 expression.

As shown in FIG. 6. treatment of stemness-high cancer cells (FaDu cancer stem cells) for 24 hours with DMSO or BBI608 (2 mM)) resulted in decreased expression of the self-renewal genes β-CATENIN, NANOG, SMO, and SOX2.

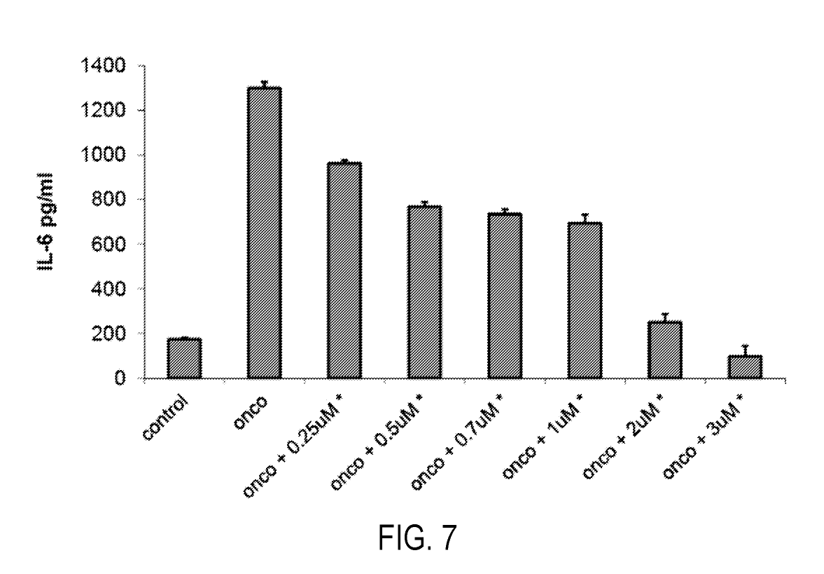

FIG. 7 shows that BBI608 downregulated IL-6 protein production by HeLa cells.

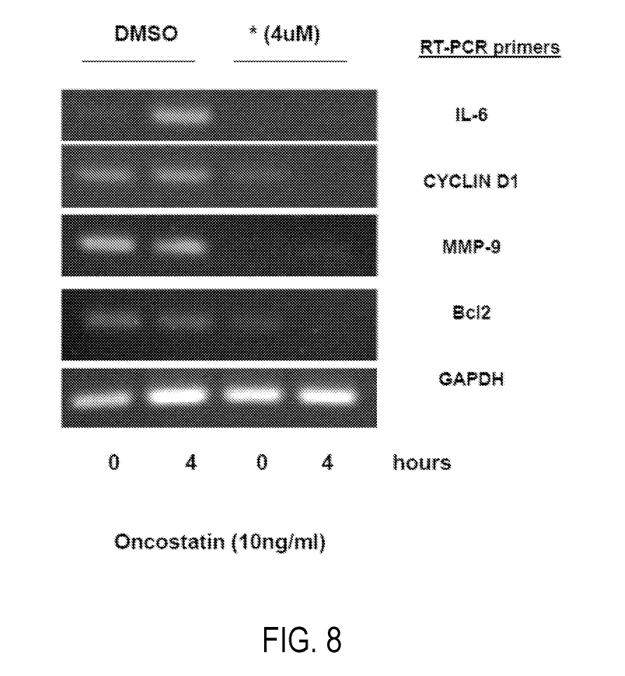

FIG. 8 shows that BBI608 downregulated IL-6 and other STAT3 target genes in HeLA cells.

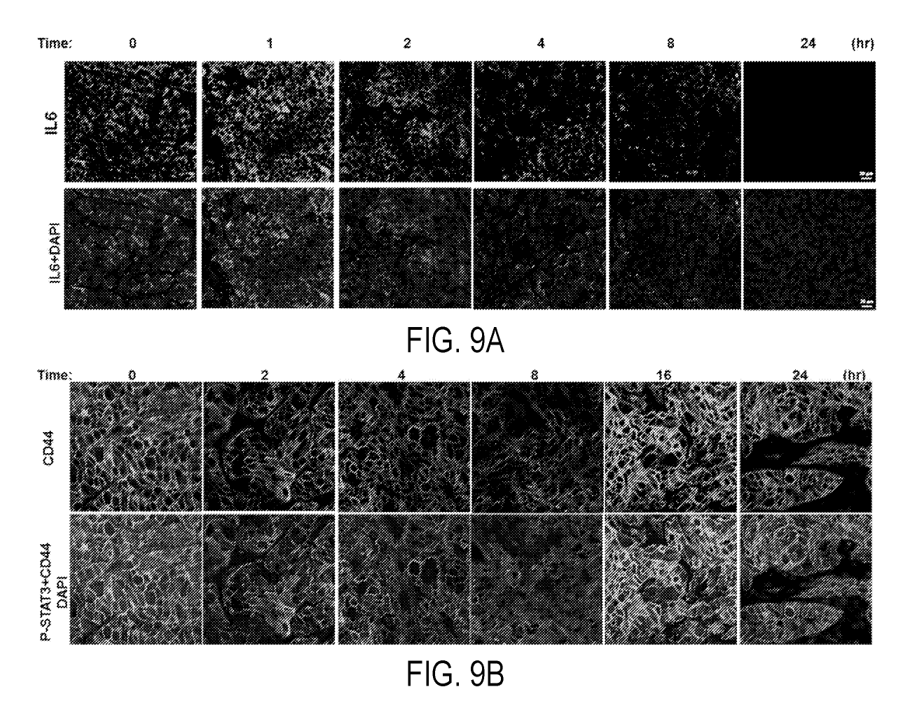

FIG. 9A shows that BBI608 reduced IL-6 level in a time-dependent manner in the colorectal cancer xenograft model (SW480).

FIG. 9B shows that BBI608 inhibited CD44 protein expression in a time-dependent manner in the ovarian cancer xenograft model (SKOV-3).

FIG. 10A shows that BBI608 reduced IDO1 protein levels in SKOV3 cells after treatment with the indicated concentrations of BBI608 for 3 hours.

FIG. 10B shows that BBI608 reduced IDO1 protein levels in SKOV3 cells treated with the indicated concentrations of BBI608 for 8 or 24 hours.

FIG. 11 shows that BBI608 inhibited endogenous IDO1 expression in SKOV3 cells after a 6 or 24 hour treatment with 1 μM or 2 μM of BBI608. Specifically, RNA was isolated, reverse transcribed, and the cDNA was used in a qPCR assay to determine the mRNA levels for IDO1. Data was normalized to GAPDH.

FIG. 12A shows that BBI608 inhibited interferon-gamma (IFNγ) induced IDO1 expression in HeLa cells. Specifically, RNA from Hela cells either untreated or treated with IFN-gamma (50 ng/ml) with or without BBI608 (2 μM) for 6 hours was isolated and reverse transcribed. The cDNA generated was then used in a qPCR assay to determine the mRNA levels for IDO1. Data was normalized to GAPDH.

FIG. 12B shows another example of BBI608's inhibition of interferon-gamma (IFNγ) induced IDO1 expression in HeLa cells. Specifically, RNA from Hela cells either untreated or IFN-gamma (50 ng/ml) treated with or without BBI608 (2 μM) for 24 hours was isolated and reverse transcribed. The cDNA was then used in a qPCR assay to determine the mRNA levels for IDO1. Data was normalized to GAPDH.

FIG. 13A show that BBI608 reduced IDO1 expression level in a time-dependent manner in a colorectal cancer xenograft model (SW480). FIG. 13B shows that BBI608 also reduced the IDO1 expression level in a time-dependent manner in an ovarian cancer xenograft model (SKOV-3).

FIG. 14 shows that PD-L1 expression in tumor cells in the CT26 model was reduced by BBI608 treatment but was increased by anti-PD-1 antibody treatment.

FIG. 15A shows that IFNγ increased the expression of PD-L1 in tumor cells and BBI608 treatment reduced IFNγ-induced PD-L1 expression.

FIG. 15B shows that administration of BBI608 resulted in the down-regulation of PD-L1 expression staining of B16F10 melanoma cells in a murine xenograft model, demonstrating the ability of BBI608 to inhibit immune evasion mechanisms in vivo.

### Example 5: Induction of a Tumor Antigen-Specific T Cell Immune Response After Treatment With BBI-608, and/or an Anti-PD1 Antibody

### Apc^{Min/+} C57BL/6 Mice

All animals were housed in Association for Assessment and Accreditation of Laboratory Animal Care-approved facilities in static microisolator cages. Testing confirmed the mice were pathogen-free and there was no evidence of murine Helicobacter spp. by culture or PCR. Apc^{Min/+} mice on a C57BL/6J background were originally obtained from the Jackson Laboratory (Bar Harbor, Me.) and bred in-house to wild-type (wt) C57BL/6J mice to generate Apc^{Min/+}. 17 week old Apc^{Min/+} mice or matched wild-type controls were treated with either vehicle alone, or BBI608 at 200 mg/kg daily by oral gavage (p.o., q.d.) for 4 consecutive days (n=4/group). Body weight and clinical signs were monitored throughout the course of the treatment. On treatment day 4, the animals were sacrificed 4 hours after the last dose and the tumors in the Apc^{Min/+} small intestine or a piece of normal intestine, from wild type controls, were harvested.

### Measurement of Tumor Antigen-Specific T Cell Immune Response

Spleen tissues were collected from the above APC^{Min/+} mice at the time of tumor harvesting. CD8⁺ T cells were separated using a CD8⁺ T cells isolation kit according to the manufacturer's protocol (STEMCELL Technology). Two large tumors were collected from control APC^{Min/+} mice, minced to ˜1 mm³ in size in RPMI cell culture medium containing 10% FBS, and irradiated with 30 Gy X-ray. Isolated spleen T cells were then cultured in vitro for 24 hours with anti-CD28 antibody in the presence or absence of irradiated Apc tumor pieces containing tumor specific antigens. Golgi secretion inhibitor monensin was added to each sample during the last 6 hours of T cells culture. After 24 hour culture, T cells were collected and stained with Alexa-488 labeled rat anti-mouse CD8a antibody (1:100, Biolegend), then fixed and permeabilized with Cytofix/Cytoperm (BD) according to the manufacturer's protocol. Intracellular IFN-γ was then stained with Alexa-647 labeled rat anti-mouse IFN-γ antibody (1:100, BD) and CD8⁺ T cells producing IFN-γ were analyzed under the Zeiss fluorescence microscope described above.

FIG. 16 shows that treatment with BBI608 resulted in a robust immune response with multiple centers of B-cell proliferation evident in the lymph node adjacent to the xenograft B16F10 tumor, demonstrating the efficacy of BBI608 at inducing a T-cell response in vivo. The tissues were stained with PCNA.

FIG. 17 shows that, in an Apc^{Min/+} mouse model of colon cancer, it was hard to find CD8⁺ T cells in the control group, but BBI608 increased the number of tumor infiltrating CD8⁺ T cells significantly. CD8⁺ T cell proliferation was demonstrated through the detection of an increased expression of the proliferation marker PCNA. CD8⁺ levels were analyzed by immunofluorescence.

FIG. 18 shows that tumor-infiltrating T cells (TILs) tended to increase with BBI608 and anti-PD-1 antibody monotherapies, although this trend was not statistically significant. However, the combination of BBI608 and anti-PD-1 antibody treatment resulted in more than a threefold increase in the number of tumor-infiltrating T cells as compared to control tumors (FIG. 18 and FIG. 19A). The treatment effects on the cytotoxic T lymphocytes (CTL) subpopulation were assessed by performing FACS analyses on cells dissociated from CT26 tumors. In order to have enough cells for analysis for the combination group, tumors were harvested after two days of treatment. Consistent with the immunofluorescence staining results, the number of tumor infiltrating T cells (CD3⁺) increased more than twofold in the BBI608 and anti-PD-1 antibody combined treatment group as compared to the control (FIG. 19B). The BBI608 and anti-PD-1 antibody treatment combination also resulted in more than a twofold increase in tumor infiltrating cytotoxic T cells (CD3⁺ and CD8⁺) as compared to the control (FIG. 19C).

FIG. 20 shows that, in the presence of the tumor antigen, a higher percentage of CD8⁺ T cells (cytotoxic T cells) from BBI608 treated samples produced INF-γ than control samples, suggesting BBI608 also increased the number of tumor-specific cytotoxic T cells.

The embodiments illustrated and discussed in this specification are intended only to teach those skilled in the art the best way known to the inventors at the time of filing to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

What is claimed is:

1. A method of treating cancer in a subject in need thereof comprising administering:
(a) a therapeutically effective amount of at least one first compound chosen from cancer stemness inhibitors, prodrugs thereof, pharmaceutically acceptable salts of any of the foregoing, and solvates of any of the foregoing; and
(b) a therapeutically effective amount of at least one second compound chosen from immunotherapeutic agents, prodrugs thereof, pharmaceutically acceptable salts of any of the foregoing, and solvates of any of the foregoing.

2. A method of treating cancer refractory or resistant to an immunotherapeutic agent in a subject comprising administering:
(a) a therapeutically effective amount of at least one first compound chosen from cancer stemness inhibitors, prodrugs thereof, pharmaceutically acceptable salts of any of the foregoing, and solvates of any of the foregoing; and
(b) a therapeutically effective amount of at least one second compound chosen from immunotherapeutic agents, prodrugs thereof, pharmaceutically acceptable salts of any of the foregoing, and solvates of any of the foregoing.

3. A method of preventing cancer relapse in a subject comprising administering:
(a) a therapeutically effective amount of at least one first compound chosen from cancer stemness inhibitors, prodrugs thereof, pharmaceutically acceptable salts of any of the foregoing, and solvates of any of the foregoing; and
(b) a therapeutically effective amount of at least one second compound chosen from immunotherapeutic agents, prodrugs thereof, pharmaceutically acceptable salts of any of the foregoing, and solvates of any of the foregoing.

4. A method of suppressing regrowth or recurrence of cancer in a subject comprising administering:
(a) a therapeutically effective amount of at least one first compound chosen from cancer stemness inhibitors, prodrugs thereof, pharmaceutically acceptable salts of any of the foregoing, and solvates of any of the foregoing; and
(b) a therapeutically effective amount of at least one second compound chosen from immunotherapeutic agents, prodrugs thereof, pharmaceutically acceptable salts of any of the foregoing, and solvates of any of the foregoing.

5. The method according to any one of claims 1-4, wherein the cancer stemness inhibitors comprise STAT3 pathway inhibitors.

6. The method according to any one of claims 1-5, wherein the cancer stemness inhibitors comprise 2-(1-hydroxyethyl)-naphtho[2,3-b]furan-4,9-dione, 2-acetyl-7-chloro-naphtho[2,3-b]furan-4,9-dione, 2-acetyl-7-fluoro-naphtho[2,3-b]furan-4,9-dione, 2-acetylnaphtho[2,3-b]furan-4,9-dione, and 2-ethyl-naphtho[2,3-b]furan-4,9-dione.

7. The method according to any one of claims 1-6, wherein the cancer stemness inhibitors comprise compounds having formula A:

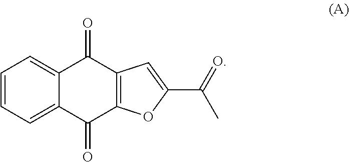 [SMILES: CC(=O)c1cc2c(o1)C(=O)c1ccccc1C2=O]

8. The method according to any one of claims 1-7, wherein the immunotherapeutic agents comprise immune checkpoint modulators.

9. The method according to any one of claims 1-8, wherein the immunotherapeutic agents comprise therapeutics targeting PD1 or PDL1 or other immune checkpoint modulation agents.

10. The method according to any one of claims 1-9, wherein the subject has an immune checkpoint gene expression level above a benchmark level.

11. The method according to claim 10, wherein the immune checkpoint gene is chosen from PD-1, PD-L1, PD-L2, CTLA-4, IDO1, STAT3, and IL-6.

12. The method according to any one of claims 1-11, wherein the subject has a cancer stemness gene expression level above a benchmark.

13. The method according to claim 12, wherein the cancer stemness gene is chosen from β-CATENIN, NANOG, SMO, SOX2, STAT3, AXL, ATM, C-MYC, KLF4, SURVIVIN, or BMI-1.

14. The method according to any one of claims 1-13, wherein the cancer is chosen from esophageal cancer, gastroesophageal junction cancer, lung cancer, gastrointestinal cancer, leukemia, lymphoma, myeloma, brain cancer, pancreatic cancer, endometrial cancer, prostate cancer, liver cancer, gastroesophageal adenocarcinoma, chondrosarcoma, colorectal adenocarcinoma, breast cancer, bladder cancer, renal cell carcinoma, ovarian cancer, head and neck cancer, melanoma, gastric adenocarcinoma, sarcoma, genitourinary cancer, gynecologic cancer, or adrenocorticoid carcinoma.

15. The method according to any one of claims 1-14, wherein the cancer is chosen from melanoma, breast cancer, bladder cancer, renal cell carcinoma, colorectal cancer, pancreatic cancer, or endometrial cancer.

16. The method according to any one of claims 1-15, wherein the cancer is advanced, refractory, recurrent, or metastatic.

17. A method of sensitizing or re-sensitizing cancer cells to an immunotherapeutic agent comprising administering to the cancer cells at least one first compound chosen from cancer stemness inhibitors, prodrugs thereof, derivatives thereof, pharmaceutically acceptable salts of any of the foregoing, and solvates of any of the foregoing.

18. The method according to claim 17, wherein the sensitization or re-sensitization of the cancer cells comprises changing the level of at least one protein chosen from proteins that are capable of assisting cancer cells to escape from the immune system.

19. The method according to claim 18, wherein the proteins comprises PD-L1, PD-L2, IDO-1, CTLA-4, and IL-6.

20. A method of increasing the number of immune cells, increasing the survival of immune cells, or activating immune cells in or around cancer cells comprising administering at least one first compound chosen from cancer stemness inhibitors, prodrugs thereof, derivatives thereof, pharmaceutically acceptable salts of any of the foregoing, and solvates of any of the foregoing.

21. The method according to any one of claims 17-20, wherein the cancer cells are in a subject.

22. The method according to any one of claims 17-21, wherein the cancer stemness inhibitors comprise STAT3 pathway inhibitors.

23. The method according to any one of claims 17-22, wherein the cancer stemness inhibitors comprise 2-(1-hydroxyethyl)-naphtho[2,3-b]furan-4,9-dione, 2-acetyl-7-chloro-naphtho[2,3-b]furan-4,9-dione, 2-acetyl-7-fluoro-naphtho[2,3-b]furan-4,9-dione, 2-acetylnaphtho[2,3-b]furan-4,9-dione, and 2-ethyl-naphtho[2,3-b]furan-4,9-dione.

24. The method according to any one of claims 17-23, wherein the cancer stemness inhibitors comprise compounds having formula A:

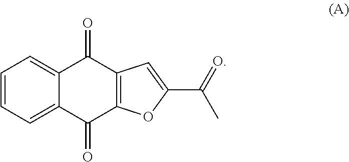 [SMILES: CC(=O)c1cc2c(o1)C(=O)c1ccccc1C2=O]

25. The method according to any one of claims 17-24, comprising administering a therapeutically effective amount of at least one second compound chosen from immunotherapeutic agents, prodrugs thereof, derivatives thereof, pharmaceutically acceptable salts of any of the foregoing, and solvates of any of the foregoing.

26. The method according to any one of claims 17-25, wherein the immunotherapeutic agents comprise immune checkpoint modulators.

27. The method according to any one of claims 17-26, wherein the immunotherapeutic agents comprise therapeutics targeting PD1 or PDL1 or other immune checkpoint modulation agents.

28. The method according to any one of claims 17-27, wherein the cancer is chosen from esophageal cancer, gastroesophageal junction cancer, lung cancer, gastrointestinal cancer, leukemia, lymphoma, myeloma, brain cancer, pancreatic cancer, endometrial cancer, prostate cancer, liver cancer, gastroesophageal adenocarcinoma, chondrosarcoma, colorectal adenocarcinoma, breast cancer, bladder cancer, renal cell carcinoma, ovarian cancer, head and neck cancer, melanoma, gastric adenocarcinoma, sarcoma, genitourinary cancer, gynecologic cancer, or adrenocorticoid carcinoma.

29. The method according to any one of claims 17-28, wherein the cancer is chosen from melanoma, breast cancer, bladder cancer, renal cell carcinoma, colorectal cancer, pancreatic cancer, or endometrial cancer.

30. The method according to any one of claims 17-29, wherein the cancer is advanced, refractory, recurrent, or metastatic.

31. The method according to any one of claims 17-30, wherein the cancer is microsatellite instability-high metastatic colorectal cancer.

32. The method according to any one of claims 17-30, wherein said cancer is microsatellite stable metastatic colorectal cancer.

33. The method according to any one of claims 17-32, wherein said cancer is with mismatch-repair deficiency.

34. The method according to any one of claims 17-32, wherein said cancer is without mismatch-repair deficiency.

35. A method of treating cancer in a subject comprising administering to a subject in need thereof a therapeutically effective amount of at least one compound of formula A chosen from compounds having formula A: 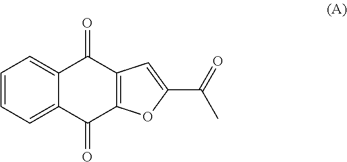 [SMILES: CC(=O)c1cc2c(o1)C(=O)c1ccccc1C2=O]
prodrugs thereof, derivatives thereof, pharmaceutically acceptable salts of any of the foregoing, and solvates of any of the foregoing; and
a therapeutically effective amount of at least one immune checkpoint modulator chosen from nivolumab, pembrolizumab, and ipilimumab.

36. A method of treating a cancer refractory or resistant to an immunotherapeutic agent in a subject comprising administering to a subject in need thereof a therapeutically effective amount of at least one compound of formula A chosen from compounds having formula A: 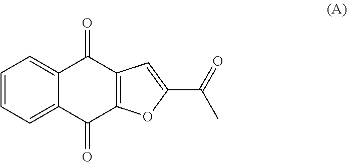 [SMILES: CC(=O)c1cc2c(o1)C(=O)c1ccccc1C2=O]
prodrugs thereof, derivatives thereof, pharmaceutically acceptable salts of any of the foregoing, and solvates of any of the foregoing; and
a therapeutically effective amount of at least one immune checkpoint modulator chosen from nivolumab, pembrolizumab, and ipilimumab.

37. A method of sensitizing a cancer to an immune response in a subject comprising administering to a subject in need thereof a therapeutically effective amount of at least one compound of formula A chosen from compounds having formula A: 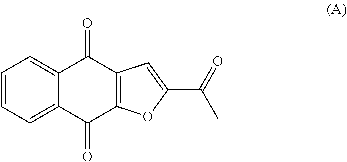 [SMILES: CC(=O)c1cc2c(o1)C(=O)c1ccccc1C2=O]
prodrugs thereof, derivatives thereof, pharmaceutically acceptable salts of any of the foregoing, and solvates of any of the foregoing.

38. A method of re-sensitizing a cancer to an immune response in a subject comprising administering to a subject in need thereof a therapeutically effective amount of at least one compound of formula A chosen from compounds having formula A: 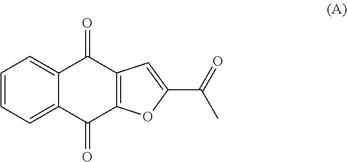 [SMILES: CC(=O)c1cc2c(o1)C(=O)c1ccccc1C2=O]
prodrugs, derivatives, pharmaceutically acceptable salts of any of the foregoing, and solvates of any of the foregoing.

39. The method according to claim 37 or claim 38, wherein the sensitizing or re-sensitizing the cancer comprises increasing the level of immune cells.

40. The method according to any one of claims 37-39, wherein the sensitizing or re-sensitizing the cancer comprises increasing the survival of immune cells.

41. The method according to any one of claims 37-40, wherein the sensitizing or re-sensitizing the cancer comprises activating immune cells.

42. The method according to any one of claims 37-41, wherein the immune cells comprise T cells.

43. The method according to claim 42, wherein the T cells comprise CD8⁺ cells.

44. The method according to any one of claims 37-41, wherein the immune cells comprise T helper cells.

45. The method according to any one of claims 37-41, wherein the immune cells comprise antigen-presenting cells.

46. The method according to claim 45, wherein the immune cells comprise dendritic cells.

47. The method according to any one of claims 37-46, wherein the sensitizing or re-sensitizing the cancer comprises reducing the expression of an immune checkpoint gene.

48. The method according to any one of claims 37-47, comprising reducing expression of IDO1.

49. The method according to any one of claims 37-48, comprising reducing expression of PD-L1.

50. The method according to any one of claims 37-49, comprising reducing expression of PD-L2.

51. The method according to any one of claims 37-50, comprising reducing expression of IL-6.

52. A kit comprising (1) at least one first compound chosen from cancer stemness inhibitors, prodrugs thereof, derivatives thereof, pharmaceutically acceptable salts of any of the foregoing, and solvates of any of the foregoing, (2) at least one second compound chosen from immunotherapeutic agents, prodrugs thereof, derivatives thereof, pharmaceutically acceptable salts of any of the foregoing, and solvates of any of the foregoing, and (3) instructions for administration and/or use of the at least one first compound and the at least one second compound.
